(19) United States
(12) Patent Application Publication
(10) Pub. No.: US 2025/0243251 A1
(43) Pub. Date: Jul. 31, 2025

(54) COMPOSITIONS AND METHODS FOR TARGETING MXRA8 TO TREAT OBESITY AND ASSOCIATED METABOLIC DISEASES

(71) Applicants: Jonathan R. Brestoff Parker, St. Louis, MO (US); Michael Diamond, St. Louis, MO (US); Wentong Jia, St. Louis, MO (US)

(72) Inventors: Jonathan R. Brestoff Parker, St. Louis, MO (US); Michael Diamond, St. Louis, MO (US); Wentong Jia, St. Louis, MO (US)

(73) Assignee: Washington University, St. Louis, MO (US)

(21) Appl. No.: 19/037,260

(22) Filed: Jan. 26, 2025

Related U.S. Application Data
(60) Provisional application No. 63/625,555, filed on Jan. 26, 2024.

(51) Int. Cl. C07K 14/47; A61K 38/00; A61K 47/64; A61P 3/04

(52) U.S. Cl. CPC C07K 14/4702; A61K 47/64; A61P 3/04; A61K 38/00; C07K 2319/30

(57) ABSTRACT

Compositions and formulations of synthetic MXRA8-Fc fusion proteins are provided, as well as methods of use thereof. Methods include neutralizing TSP1/MXRA8 interactions in a subject by administering a synthetic MXRA8-Fc fusion protein, such that the synthetic MXRA8-Fc fusion protein binds to TSP1 to inhibit the TSP1 from binding with MXRA8. Methods also include treating obesity such as high fat diet (HFD)-induced obesity, and treating obesity-associated metabolic diseases such as type 2 diabetes by administering a synthetic MXRA8-Fc fusion protein.

### CROSS-REFERENCE TO RELATED APPLICATIONS

This application claims the benefit of priority to U.S. Provisional Application Ser. No. 63/625,555 filed on Jan. 26, 2024, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD

The present disclosure generally relates to new fusion protein compositions and methods of use thereof for treating obesity and associated metabolic diseases.

### BACKGROUND

MXRA8 is the receptor for Chikungunya virus and has no clear function in mammalian physiology.

### SUMMARY

Among the various aspects of the present disclosure is the provision of new MXRA8-Fc fusion protein compositions, as well as methods of use thereof for treating obesity and associated metabolic diseases.

In an aspect of the present disclosure, a composition is provided. The composition comprises a synthetic MXRA8-Fc fusion protein or pharmaceutically acceptable salt thereof. In some embodiments, the synthetic MXRA8-Fc fusion protein comprises an immunoglobulin (IgG) Fc domain and an MXRA8 protein, further comprises at least one of a pharmaceutically acceptable excipient and a pharmaceutically acceptable carrier, and/or further comprises protein A-coated beads attached to the synthetic MXRA8-Fc fusion protein.

In another aspect of the present disclosure, a method of treating a metabolic associated disease in a subject in need thereof is provided. The method comprises: administering to the subject a composition comprising a synthetic MXRA8-Fc fusion protein or pharmaceutically acceptable salt thereof. In some embodiments, administering the composition neutralizes TSP1/MXRA8 interactions in the subject and/or the synthetic MXRA8-Fc fusion protein comprises an immunoglobulin (IgG) Fc domain and an MXRA8 protein. In some embodiments of the method, the composition further comprises at least one of a pharmaceutically acceptable excipient and a pharmaceutically acceptable carrier and/or further comprises protein A-coated beads attached to the synthetic MXRA8-Fc fusion protein. In some embodiments, the metabolic associated disease is a high fat diet (HFD)-induced disease. Depending on the embodiment, the HFD-induced disease is at least one of type 2 diabetes and fatty liver disease.

In a further aspect of the present disclosure, a method of treating obesity in a subject in need thereof is provided. The method comprises: administering to the subject a composition comprising a synthetic MXRA8-Fc fusion protein or pharmaceutically acceptable salt thereof. In some embodiments, administering the composition neutralizes TSP1/MXRA8 interactions in the subject and/or the synthetic MXRA8-Fc fusion protein comprises an immunoglobulin (IgG) Fc domain and an MXRA8 protein. In some embodiments of the method, the composition further comprises at least one of a pharmaceutically acceptable excipient and a pharmaceutically acceptable carrier and/or further comprises protein A-coated beads attached to the synthetic MXRA8-Fc fusion protein. In some embodiments, the obesity is high fat diet (HFD)-induced obesity. In some embodiments, the subject has type 2 diabetes and/or fatty liver disease.

In accordance with other aspects of the present disclosure, a composition comprising a synthetic MXRA8-Fc fusion protein or pharmaceutically acceptable salt thereof is provided. In one embodiment, the synthetic MXRA8-Fc fusion protein binds to soluble TSP1 to inhibit TSP1 from binding with MXRA8.

In accordance with additional aspects of the present disclosure, a formulation is provided. The formulation comprises: a biologically active agent comprising a synthetic MXRA8-Fc fusion protein or pharmaceutically acceptable salt thereof; and a biologically inert agent comprising at least one of a pharmaceutically acceptable excipient and a pharmaceutically acceptable carrier. In one embodiment, the synthetic MXRA8-Fc fusion protein binds to soluble TSP1 to inhibit TSP1 from binding with MXRA8.

In accordance with yet other aspects of the present disclosure, a method for treating a metabolic associated disease in a subject, the method comprising neutralizing TSP1/MXRA8 interactions in the subject. In one embodiment, treating the metabolic associated disease comprising treating a high fat diet (HFD)-induced disease. In another embodiment, the HFD-induced disease is selected from obesity and type 2 diabetes. In another embodiment, the method of neutralizing TSP1/MXRA8 interactions further comprising administering a therapeutically effective amount of a TSP1/MXRA8 inhibiting agent. In another embodiment, the TSP1/MXRA8 inhibiting agent is a synthetic MXRA8-Fc fusion protein which binds to TSP1. In yet another embodiment, the TSP1 is soluble TSP1.

In accordance with yet additional aspects of the present disclosure, a method for neutralizing TSP1/MXRA8 interactions in a subject is provided. The method comprises administering a synthetic MXRA8-Fc fusion protein to the subject, wherein the synthetic MXRA8-Fc fusion protein binds to TSP1 to inhibit the TSP1 from binding with MXRA8. In one embodiment, the TSP1 is soluble TSP1. In one embodiment, the subject has a metabolic associated disease. In another embodiment, the metabolic associated disease is induced by a high fat diet (HFD). In another embodiment, the HFD-induced metabolic associated disease is selected from obesity and type 2 diabetes.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a chart showing MXRA8 expression in different organ systems using the Human Protein Atlas (HPA) dataset, wherein MXRA8 is highly expressed in adipose tissue.

FIG. 1B is a chart showing MXRA8 expression in different organ systems using the Genotype-Tissue Expression (GTEx) dataset, wherein MXRA8 is highly expressed in adipose tissue.

FIG. 2A is a graph of MXRA8 transcript levels in subcutaneous white adipose tissue (WAT) from metabolically normal lean (MNL), metabolically normal obese (MNO), and metabolically abnormal obese (MAO) people.

FIG. 2B is a graph of MXRA8 expression in lean (dark grey) and obese (light grey) males (left) and females (right).

FIG. 2C is a scatterplot showing a positive correlation between increased MXRA8 expression and an increase in body mass index (BMI), p=0.003.

FIG. 2D is a scatterplot showing a positive correlation between increased MXRA8 expression and percent whole-body adiposity (p=0.002).

FIG. 2E is a scatterplot showing a positive correlation between MXRA8 expression and subcutaneous adipose tissue volume (p=0.002).

FIG. 2F is a scatterplot showing a positive correlation between MXRA8 expression and intra-abdominal adipose tissue volume (p=0.003).

FIG. 2G is a scatterplot showing a positive correlation between MXRA8 expression and percent of liver fat (p<0.001).

FIG. 2H is a scatterplot showing no correlation between MXRA8 expression and lean mass (p=0.072).

FIG. 2I is a scatterplot showing no correlation between MXRA8 expression and bone mass (p=0.5).

FIG. 2J is a graph of MXRA8 expression in the WAT of euglycemic (dark grey) and hyperglycemic (light grey) patients.

FIG. 2K is a scatterplot showing a positive correlation between WAT MXRA8 expression and glucose tolerance (p<0.001). GTT AUC; glucose tolerance test area under the curve.

FIG. 2L is a scatterplot showing a negative correlation between MXRA8 expression and insulin resistance (p<0.01). M/I; glucose metabolism/insulin concentration.

FIG. 3A is a graph of MXRA8 transcript levels in subcutaneous WAT from lean (dark grey) and obese (light grey) patients of different ethnicities (White, Black/African American, Asian/Pacific Islander).

FIG. 3B is a scatterplot showing no correlation between MXRA8 expression and age (p=0.146).

FIG. 4A is a scatterplot showing no correlation between MXRA8 expression and the total cholesterol (mg/dL) (p=0.802).

FIG. 4B is a scatterplot showing no correlation between MXRA8 expression and LDL cholesterol (p=0.272).

FIG. 4C is a scatterplot showing a negative correlation between MXRA8 expression and HDL cholesterol (p=0.004).

FIG. 4D is a scatterplot showing no correlation between MXRA8 expression and triglycerides (mg/dL) (p=0.115).

FIG. 4E is a scatterplot showing no correlation between MXRA8 expression and serum free fatty acids (FFA; mM) (p=0.6567).

FIG. 4F is a scatterplot showing a positive correlation between MXRA8 and basal glucose (mg/dL) (p=0.004).

FIG. 4G is a scatterplot showing a positive correlation between MXRA8 expression and basal insulin (IU/mL) (p<0.001).

FIG. 5A is a graph of Mxra8 mRNA expression in murine epididymal (e)WAT, inguinal (i)WAT, brown adipose tissue (BAT), and spleen.

FIG. 5B is a western blot showing MXRA8 protein expression in eWAT, iWAT, and BAT from wildtype mice fed a high fat diet (HFD) or a normal chow diet (NCD) for 10 weeks.

FIG. 5C is a graph of MXRA8 protein level (quantified from the western blot in FIG. 5B) in eWAT, iWAT, and BAT from mice fed a HFD or NCD for 10 weeks.

FIG. 5D is a graph of Mxra8 mRNA expression from eWAT adipocytes and stromal vascular fraction (FVF) cells from wildtype mice fed a NCD.

FIG. 5E is a UMAP of mouse Mxra8 expression in SVF cells, including adipocyte progenitor cells (APCs), adipocytes, and mesothelium. The data is from a publicly available single nucleus RNA sequencing (snRNAseq) dataset (GSE176171).

FIG. 5F is a UMAP of human Mxra8 expression in SVF cells, including adipocyte progenitor cells (APCs), adipocytes, and mesothelium. The data is from a publicly available single nucleus RNA sequencing (snRNAseq) dataset (GSE176171).

FIG. 5G is a set of flow cytometry plots of eWAT SVF from wildtype and MXRA8-deficient mice (Mxra8d8/d8). Single eWAT cells were sorted into T cells (top; middle) and APCs (top; right). MXRA8+ cells were present in the APCs (bottom, right) but not in T cells (bottom, left).

FIG. 5H is a set of flow cytometry plots showing the expression of MXRA8 in CD45− CD11 b− PDGFRa+ cells from eWAT (left), iWAT (middle), and BAT (right) cells from mice fed an NCD (top) or HFD (bottom).

FIG. 5I is a graph of MXRA8+ APCs from eWAT, iWAT, and BAT from mice fed a NCD or HFD. The graph quantified the flow cytometry data from FIG. 5H.

FIG. 6A is a graph of the body weight of wildtype and Mxra8d8/d8 (Δ8) mice fed a NCD.

FIG. 6B is a graph of lean mass and fat mass of wildtype and Mxra8d8/d8 (Δ8) mice fed a NCD.

FIG. 6C is a graph of percent adiposity of wildtype and Mxra8d8/d8 (Δ8) mice fed a NCD.

FIG. 6D is a graph of energy expenditure, measured as heat (kcal/kg/hr) of wildtype and Mxra8d8/d8 (Δ8) mice fed a NCD. Measurements were taken over 24 hours, with 12 hours of light (white) and 12 hours of dark (grey).

FIG. 6E is a graph of energy expenditure, measured as heat (kcal/kg/hr) of wildtype and Mxra8d8/d8 (Δ8) mice fed a NCD. Measurements were taken during the light phase or dark phase of a light/dark cycle.

FIG. 6F is a graph of respirator exchange ratios (RER) of wildtype and Mxra8d8/d8 (Δ8) mice fed a NCD. Measurements were taken during the light phase or dark phase of a light/dark cycle.

FIG. 6G is a graph of mouse activity of wildtype and Mxra8d8/d8 (Δ8) mice fed a NCD. Measurements were taken during the light phase or dark phase of a light/dark cycle.

FIG. 6H is a graph of food intake of wildtype and Mxra8d8/d8 (Δ8) mice fed a NCD. Measurements were taken during the light phase or dark phase of a light/dark cycle.

FIG. 7A is a graph Mxra8 mRNA expression in eWAT, iWAT, and BAT of wildtype and Mxra8d8/d8 (Δ8) mice.

FIG. 7B is a graph of body weight of wildtype and Mxra8d8/d8 (Δ8) mice fed a HFD over 12 weeks.

FIG. 7C is a graph of lean mass and fat mass of wildtype and Mxra8d8/d8 (Δ8) mice after 12 weeks of a HFD.

FIG. 7D is a graph of percent adiposity of wildtype and Mxra8d8/d8 (Δ8) mice after 12 weeks of a HFD.

FIG. 7E is a graph fat mass (eWAT, iWAT, BAT) of wildtype and Mxra8d8/d8 (Δ8) mice challenged with a HFD.

FIG. 7F is a set of H&E images of eWAT (left), iWAT (middle), and BAT (right) from wildtype (top) and Mxra8d8/d8 (Δ8; bottom) mice fed a HFD. eWAT from WT mice show numerous crown-like structures (CLS) which are not seen in Δ8 mice

FIG. 7G is a graph quantifying the number of immune cells present in eWAT from wildtype and Mxra8d8/d8 (Δ8) mice fed a HFD. High-dimensional spectral flow cytometric analysis identified CD45+ cells, macrophages, monocytes, dendritic cells, neutrophils, eosinophils, ILC2, NK cells, T cells, and B cells.

FIG. 7H is a graph of the number of APCs per gram of eWAT from wildtype and Mxra8d8/d8 (Δ8) mice fed a HFD.

FIG. 7I is a set of flow cytometry plots of eWAT APCs from wildtype (left) and Mxra8d8/d8 (Δ8; right) mice. APCs are classified as Ly6C+CD9−, Ly6C+CD9+, Ly6C−CD9+, Ly6C−CD9−.

FIG. 7J is a graph quantifying the number of eWAT APCs from the flow cytometry plots of FIG. 7I.

FIG. 7K is a set of Masson's Trichrome stained histological images of eWAT (left), iWAT (middle), and BAT (right) from wildtype (top) and Mxra8d8/d8 (Δ8; bottom) mice fed a HFD.

FIG. 7L is a graph of intraperitoneal glucose tolerance (IPGTT) of WT and Δ8 mice fed a HFD.

FIG. 8A is a graph comparing liver mass of WT and Δ8 mice fed a HDF.

FIG. 8B is a graph of liver lean mass and fat mass from WT and Δ8 mice fed a HFD.

FIG. 8C is a graph of percent adiposity of the liver from WT and Δ8 mice fed a HFD.

FIG. 8D is a set of H&E images of the liver from WT and Δ8 fed a HFD.

FIG. 9 is a set of flow cytometry plots identifying the immune cells present in eWAT from wildtype and Mxra8d8/d8 (Δ8) mice fed a HFD. Immune cell number are quantified in FIG. 7G.

FIG. 10 is a set of histological images of eWAT primary adipocytes from WT and Δ8 mice stained with Oil Red O.

FIG. 11A is a graph of energy expenditure, measured as heat (kcal/kg/hr) of wildtype and Mxra8d8/d8 (Δ8) mice fed a HFD. Measurements were taken over 24 hours, with 12 hours of light (white) and 12 hours of dark (grey).

FIG. 11B is a graph of energy expenditure, measured as heat (kcal/kg/hr) of wildtype and Mxra8d8/d8 (Δ8) mice fed a HFD. Measurements were taken over 24 hours, with 12 hours of light (white) and 12 hours of dark (grey).

FIG. 11C is a graph of respirator exchange ratios (RER) of wildtype and Mxra8d8/d8 (Δ8) mice fed a HFD. Measurements were taken during the light phase or dark phase of a light/dark cycle.

FIG. 11D is a graph of mouse activity of wildtype and Mxra8d8/d8 (Δ8) mice fed a HFD. Measurements were taken during the light phase or dark phase of a light/dark cycle.

FIG. 11E is a graph of food intake of wildtype and Mxra8d8/d8 (Δ8) mice fed a HFD. Measurements were taken during the light phase or dark phase of a light/dark cycle.

FIG. 11F is an image of a western blot measuring UCP1 protein levels in iWAT and BAT of wildtype and Mxra8d8/d8 (Δ8) mice fed a HFD.

FIG. 11G is a set of histological images, stained for UCP1, of iWAT (top) and BAT (bottom) from wildtype (left) and Mxra8d8/d8 (Δ8; right) mice fed a HFD.

FIG. 11H is a graph of percent weight gain of wildtype, Δ8, Ucp1^{-/-}, and Δ8/Ucp1^{-/-} double knockout mice fed a HFD over 10 weeks.

FIG. 11I is a graph of lean mass (left) and fat mass (right) of wildtype, Δ8, Ucp1^{-/-}, and Δ8/Ucp1^{-/-} mice fed a HFD.

FIG. 11J is a graph of eWAT mass of wildtype, Δ8, Ucp1^{-/-}, and Δ8/Ucp1^{-/-} mice fed a HFD.

FIG. 11K is a graph of iWAT mass of wildtype, Δ8, Ucp1^{-/-}, and Δ8/Ucp1^{-/-} mice fed a HFD.

FIG. 11L is a graph of BAT mass of wildtype, Δ8, Ucp1^{-/-}, and Δ8/Ucp1^{-/-} mice fed a HFD.

FIG. 12A is a graph showing immunoprecipitated proteins bound to MXRA8 and identified by mass spectrometry. A synthetic MXRA8-Fc fusion protein was generated and attached it to protein A-coated Dynabeads. The MXRA8-coated beads were incubated with serum from MXRA8-deficient mice fed a HFD to immunoprecipitate bound proteins. The anti-angiogenic protein Thrombospondin-1 (TSP1) strongly binds MXRA8.

FIG. 12B is an image of a western blot of TSP1 (top) and MXRA8 (bottom). TSP1 was immunoprecipitated from WT or MXRA8-deficient 3T3 fibroblasts and MXRA8 was pulled down with TSP1.

FIG. 12C is an image of a flow cytometry events (left) and corresponding graph (right). Recombinant mouse TSP1 (rmTSP1) was labeled with the fluorescent dye Cy5 and mixed with WT and MXRA8-deficient cells. rmTSP1 binds to WT cells with a higher fluorescent intensity.

FIG. 12D is an image of a western blot of WT (left) and MXRA8-deferent (right) cells treated with increasing concentrations of rmTSP1 to determine TSP1 induced signaling to p38 MAP kinase.

FIG. 12E is a set of immunohistochemistry images of BAT from WT and Δ8 stained for CD31.

FIG. 12F is a graph of relative mRNA expression of Vegfa (left) and Vegfr2 (right) from WT and Δ8 mice.

FIG. 12G is a graph of body weight of wildtype mice fed a HFD and treated with a MXRA8-Fc fusion protein or an IgG control.

FIG. 12H is a graph of BAT mass of wildtype mice fed a HFD and treated with a MXRA8-Fc fusion protein or an IgG control.

FIG. 12I is a graph of core body temperature of wildtype mice fed a HFD and treated with a MXRA8-Fc fusion protein or an IgG control.

FIG. 12J is a graph of insulin resistance of wildtype mice fed a HFD and treated with a MXRA8-Fc fusion protein or an IgG control.

FIG. 12K is a set of immunohistochemistry images of BAT, stained for CD31, from WT mice fed a HFD and treated with a MXRA8-Fc fusion protein or an IgG control.

### DETAILED DESCRIPTION

The present disclosure is based, at least in part, on the discovery that the Matrix remodeling associated 8 (MXRA8) regulates obesity pathogenesis.

MXRA8 is the receptor for Chikungunya virus and has no clear function in mammalian physiology. MXRA8 is most highly expressed in white adipose tissue (WAT) and is unregulated in obesity in both mice and humans. MXRA8-deficient mice are protected from developing obesity by preserving UCP1+ adipocytes, which increase energy expenditure to limit weight gain. MXRA8's natural endogenous ligand is Thrombospondin-1 (TSP1), a circulating protein that disrupts the vascular bed. Administering a novel MXRA8-Fc fusion protein to mice protects them diet-induced obesity. In summary, the present disclosure has established that MXRA8 is a regulator of obesity pathogenesis and associated glucose intolerance and indicate that therapeutically targeting MXRA8-TSP1 interactions with a biologic drug comprised of an effective dose of MXRA8-Fc fusion protein is a therapy for obesity and associated metabolic diseases (e.g., type 2 diabetes and fatty liver disease).

### Inhibiting Agent

One aspect of the present disclosure provides for targeting of TSP1 and/or MXRA8, its receptor, or its downstream signaling. The present disclosure provides methods of treating or preventing obesity and associated metabolic diseases based on neutralization of TSP1/MXRA8 interactions.

As described herein, inhibitors or antagonists (e.g., antibodies, fusion proteins, small molecules) can reduce or prevent obesity and associated metabolic diseases. An inhibiting agent can be any agent that can inhibit activity and/or signaling, downregulate protein level and/or expression, and/or knockdown gene expression.

As an example, an inhibiting agent can inhibit signaling.

For example, the inhibiting agent can be an antibody. As an example, the antibody can be a murine antibody, a humanized murine antibody, or a human antibody. As another example, the inhibiting agent can be an R-type antibody, wherein the R-type antibody prevents binding to a receptor, or prevents activation and downstream signaling.

As another example, the inhibiting agent can be a fusion protein. For example, the fusion protein can be a decoy receptor, such as for TSP1. Furthermore, the fusion protein can comprise a mouse or human Fc antibody domain fused to an R1 ectodomain of interest.

As another example, an inhibiting agent can be an antagonist inhibitory protein. For example, the inhibiting agent can be a viral antagonizing protein.

As another example, an inhibiting agent can be a short hairpin RNA (shRNA) or a short interfering RNA (siRNA).

As another example, an inhibiting agent can be a single guide RNA (sgRNA).

Methods for preparing an inhibiting agent (e.g., an agent capable of signal inhibition) can comprise construction of a protein/Ab scaffold containing the natural receptor as a neutralizing agent; developing inhibitors of the receptor “down-stream”; or developing inhibitors of production “up-stream”.

Inhibition can be performed by genetically modification in a subject or genetically modifying a subject to reduce or prevent expression of a gene, such as through the use of CRISPR-Cas9 or analogous technologies.

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Chemical Agent

Examples of synthetic MXRA8-Fc fusion proteins are described herein, including a pharmaceutically acceptable salt, solvate, polymorph, tautomer, prodrug, analog, or stereoisomer thereof or optionally substituted analog thereof.

The formulas, analogs, and R groups can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀ alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, includes a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C=O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O— propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O— CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Fusion Protein

The novel fusion proteins disclosed herein are recombinant proteins that include an Fc domain and a target protein sequence as known to those of skill in the art. In exemplary embodiments of the present disclosure, the Fc domain is an immunoglobulin (IgG) Fc domain (or an appropriate alternative known to those of skill in the art), and the target protein sequence comprises an MXRA8 protein sequence (or a protein sequence having at least 90% sequence identity with MXRA8). In general, fusing the Fc domain with at least one target protein and/or target peptide, a resulting fusion protein construct exhibits advantageous properties and functions such as enhanced protein stability and extended protein half-life (e.g., in vivo serum half-life) via stability of the Fc region and the Fc receptor interactions in the immune system. Extended half-life of a fusion protein administered in a therapeutic treatment context can reduce drug dosage and administration frequency, also minimizing the potential for side effects and toxicity issues. According to the present disclosure, a fusion protein comprises an Fc region (such as the IgG Fc domain) and target region including a biologically active target protein molecule (such as MXRA8). The Fc domain is known to specifically bind protein A to simplify the Fc fusion protein purification process and which further benefits development and preparation of recombinant proteins such as the fusion protein of the present disclosure. In some embodiments, the fusion protein includes a hinge region between the Fc region and the target region. In exemplary embodiments, the fusion protein forms stable dimers through disulfide bonds in the hinge region. In some embodiments, the fusion protein includes a peptide linker between the Fc region and the target region. In exemplary embodiments, the fusion protein comprises both a hinge region and a peptide linker between the Fc region and the target region, such that the fusion protein sequence comprises the following series arrangement: Fc region-hinge region-peptide linker-target region. Depending upon the embodiment, the fusion protein (or fusion protein dimer) may have a generally linear, folded, hexameric complex, or other configuration.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of a significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates the target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged | D E |
| (Acidic): | |

Conservative Subsitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tₘ of specific internal domains of the siRNA, siRNA length, position of the target sequence within the CDS (coding region), and nucleotide content of the 3′ overhangs.

### Genome Editing

As described herein, signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage by genome editing can result in protection from obesity and associated metabolic diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activate (e.g., CRISPRa), upregulate, overexpress, downregulate).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. There has recently been an improved landscape for gene therapies. For example, in the first quarter of 2019, there were 372 ongoing gene therapy clinical trials (Alliance for Regenerative Medicine, 5 Sep. 19).

Any vector known in the art can be used. For example, the vector can be a viral vector selected from retrovirus, lentivirus, herpes, adenovirus, adeno-associated virus (AAV), rabies, Ebola, lentivirus, or hybrids thereof. Some gene therapy strategies include:

| | Strategy |
|---|---|
| Viral Vectors | |
| Retroviruses | Retroviruses are RNA viruses transcribing their single- |
| | stranded |
| | genome into a double-stranded DNA copy, which can |
| | integrate into host chromosome |
| Adenoviruses | Ad can transfect a variety of quiescent and proliferating |
| (Ad) | cell types from various species and can mediate |
| | robust gene expression |
| Adeno- | Recombinant AAV vectors contain no viral DNA and can |
| associated | carry ~4.7 kb of foreign transgenic material. They |
| Viruses | are replication defective and can replicate only while |
| (AAV) | coinfecting with a helper virus |
| Non-viral | |
| vectors | |
| plasmid | pDNA has many desired characteristics as a gene |
| DNA | therapy vector; there are no limits on the size or genetic |
| (pDNA) | constitution of DNA, it is relatively inexpensive to |
| | supply, and unlike viruses, antibodies are not generated |
| | against DNA in normal individuals |
| RNAi | RNAi is a powerful tool for gene specific silencing |
| | that could be useful as an enzyme reduction therapy or |
| | means to promote read-through of a premature stop |
| | codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug composition in a form suitable for administration to a subject, such as a human. Thus, a “formulation” is a composition specifically indicating inclusion of pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing obesity and associated metabolic diseases in a subject in need of administration of a therapeutically effective amount of synthetic MXRA8-Fc fusion protein.

Methods described herein are generally performed on a subject in need thereof. A subject in need of the therapeutic methods described herein can be a subject having, diagnosed with, suspected of having, or at risk for developing obesity and associated metabolic diseases. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of synthetic MXRA8-Fc fusion protein is, for example, an amount that would cause the desired therapeutic effect in a subject while minimizing undesired side effects. In various embodiments, an effective amount of synthetic MXRA8-Fc fusion protein described herein can substantially inhibit, slow the progress of, or limit the development of symptoms and effects obesity and associated metabolic diseases.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of synthetic MXRA8-Fc fusion protein can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat, prevent, or revers obesity and associated metabolic diseases.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of synthetic MXRA8-Fc fusion protein can occur as a single event or over a time course of treatment. For example, synthetic MXRA8-Fc fusion protein can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for obesity and associated metabolic diseases.

A synthetic MXRA8-Fc fusion protein can be administered simultaneously or sequentially with another agent, such as an antibiotic, an anti-inflammatory, or another agent. For example, a synthetic MXRA8-Fc fusion protein can be administered simultaneously with another agent, such as an antibiotic or an anti-inflammatory. Simultaneous administration can occur through administration of separate compositions, each containing one or more of a synthetic MXRA8-Fc fusion protein, an antibiotic, an anti-inflammatory, or another agent. Simultaneous administration can occur through administration of one composition containing two or more of a synthetic MXRA8-Fc fusion protein, an antibiotic, an anti-inflammatory, or another agent. A synthetic MXRA8-Fc fusion protein can be administered sequentially with an antibiotic, an anti-inflammatory, or another agent. For example, a synthetic MXRA8-Fc fusion protein can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the synthetic MXRA8-Fc fusion protein may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg.

In some embodiments, a synthetic MXRA8-Fc fusion protein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—MXRA8 Binds TSP1 to Disrupt the Vascular Bed in White Adipose Tissue and Promote Obesity Pathogenesis

Matrix remodeling associated 8 (MXRA8) is a poorly understood transmembrane protein with two extracellular domains and short intracellular tail. The physiologic functions of MXRA8 are not known, however, herein it was found that MXRA8 is highly expressed in white adipose tissue and is upregulated in obesity in both mice and humans. MXRA8 is expressed predominantly by adipocyte progenitor cells and, to a lesser extent, mature adipocytes, and binds to the anti-angiogenic protein Thrombospondin (TSP1) to dysregulate and rarefy the vascular bed in WAT in obesity. As shown herein, targeting MXRA8 is an effective therapeutic to treat obesity and associated metabolic diseases, such as type 2 diabetes.

### Results

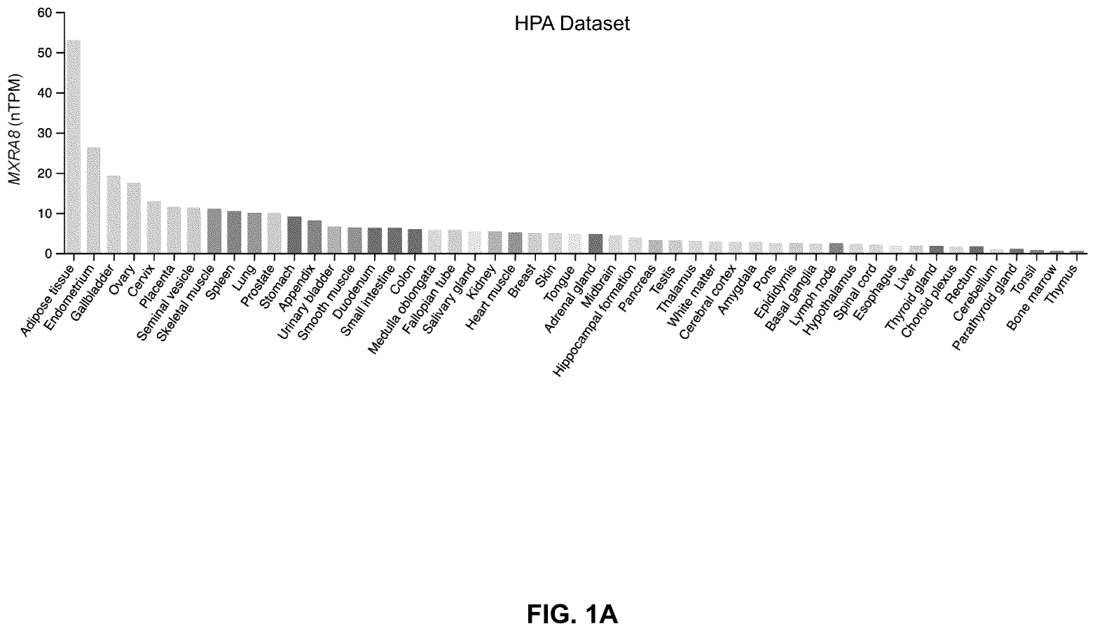

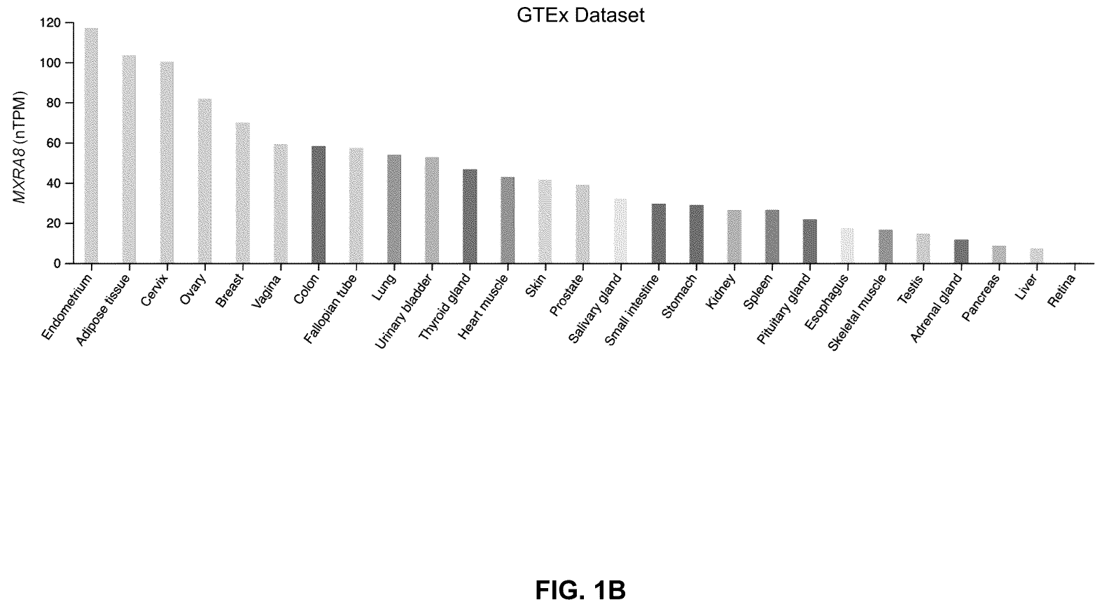

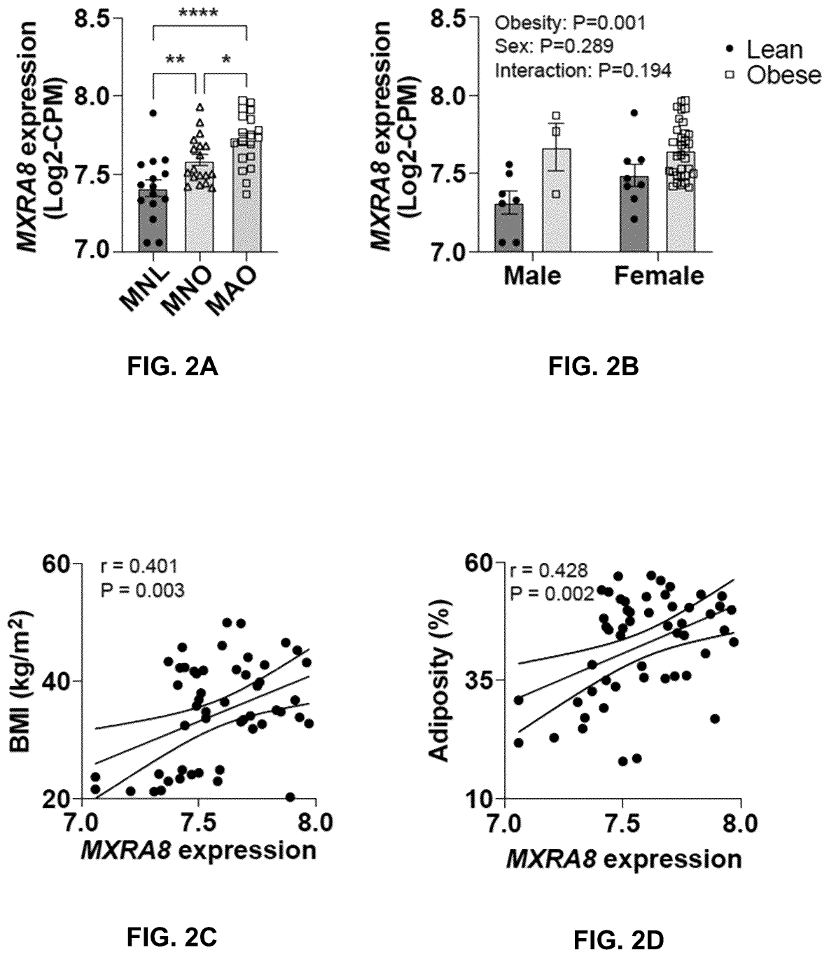

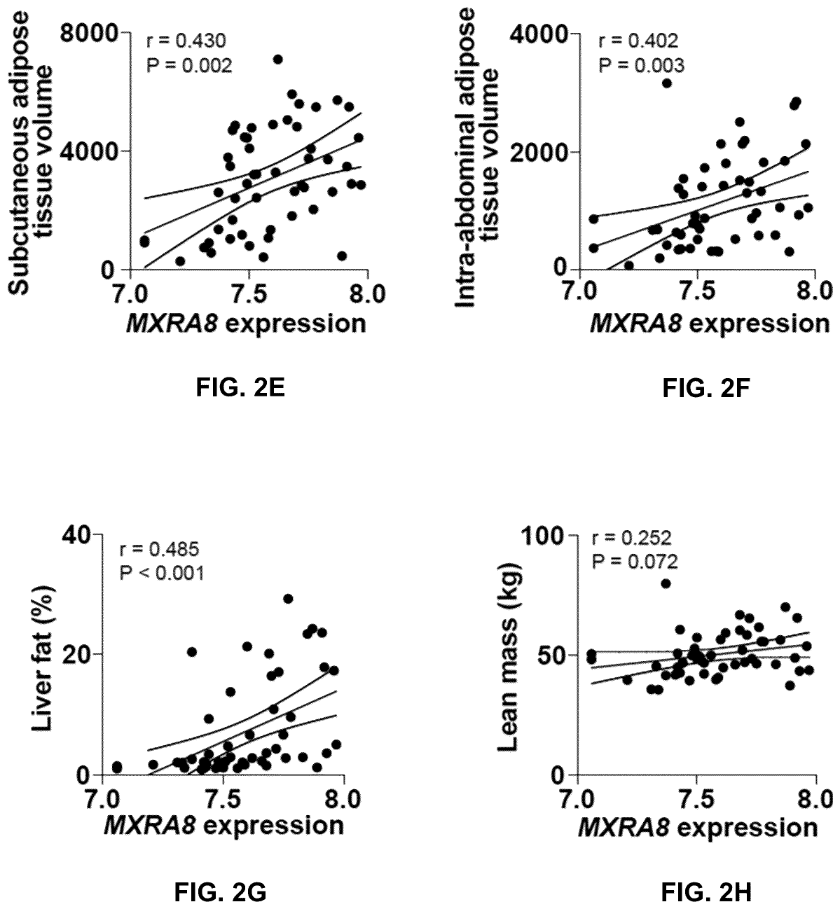

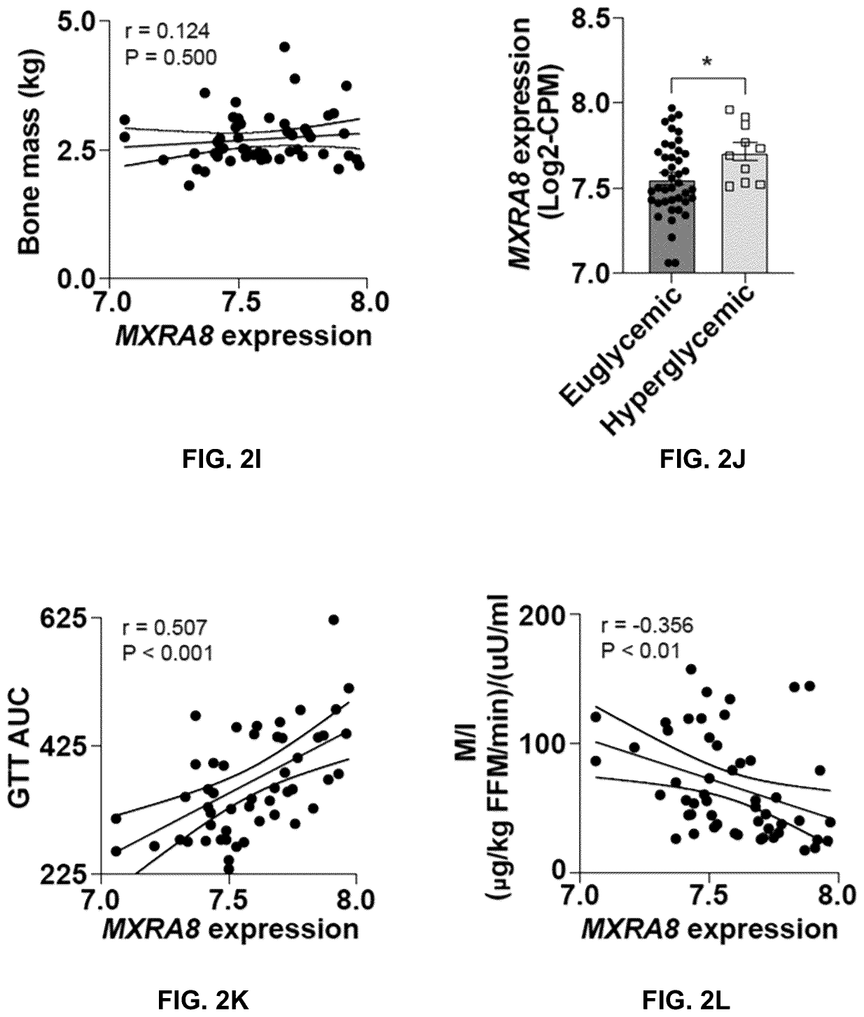

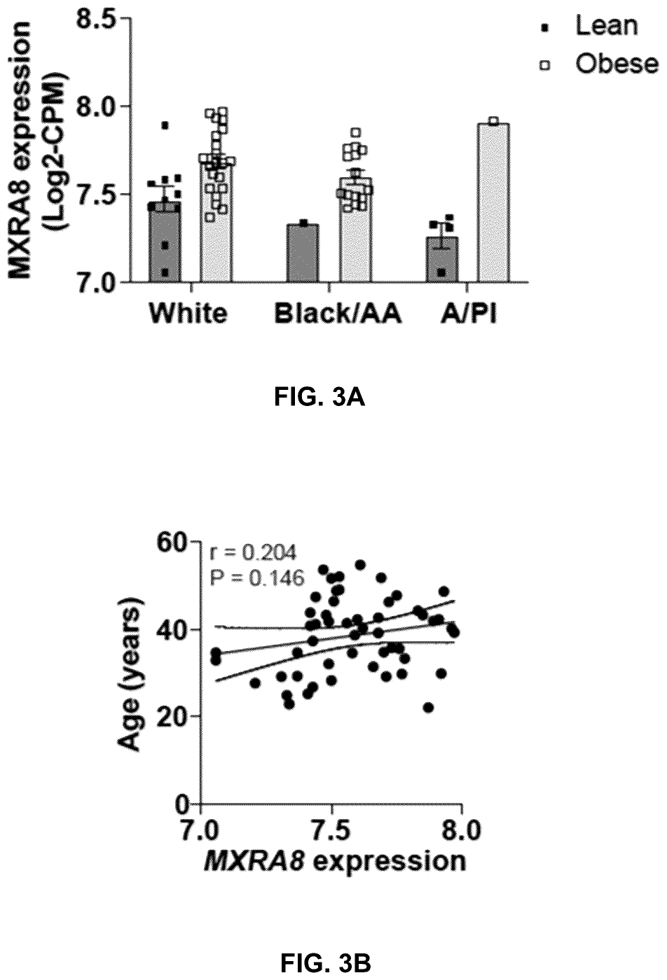

To identify the organ systems where MXRA8 is most highly expressed, the Human Protein Atlas (HPA, FIG. 1A) and GTEx (FIG. 1B) datasets were examined and it was found that adipose tissue was ranked first or second, respectively. Based on this finding, MXRA8 transcript levels in subcutaneous white adipose tissue (WAT) were compared to metabolically normal lean (MNL, n=15), metabolically normal obese (MNO, n=18), and metabolically abnormal obese (MAO, n=19) people. MXRA8 expression was found to be significantly increased in MNO and MAO groups compared to MNL controls (FIG. 2A). The increase in MXRA8 expression in WAT from obese people was observed in both males and females (FIG. 2B) and in individuals identifying as white, Black/African American, or Asian/Pacific Islanders (FIG. 3A). Age was not significantly correlated with MXRA8 expression in WAT in this cohort (FIG. 3B).

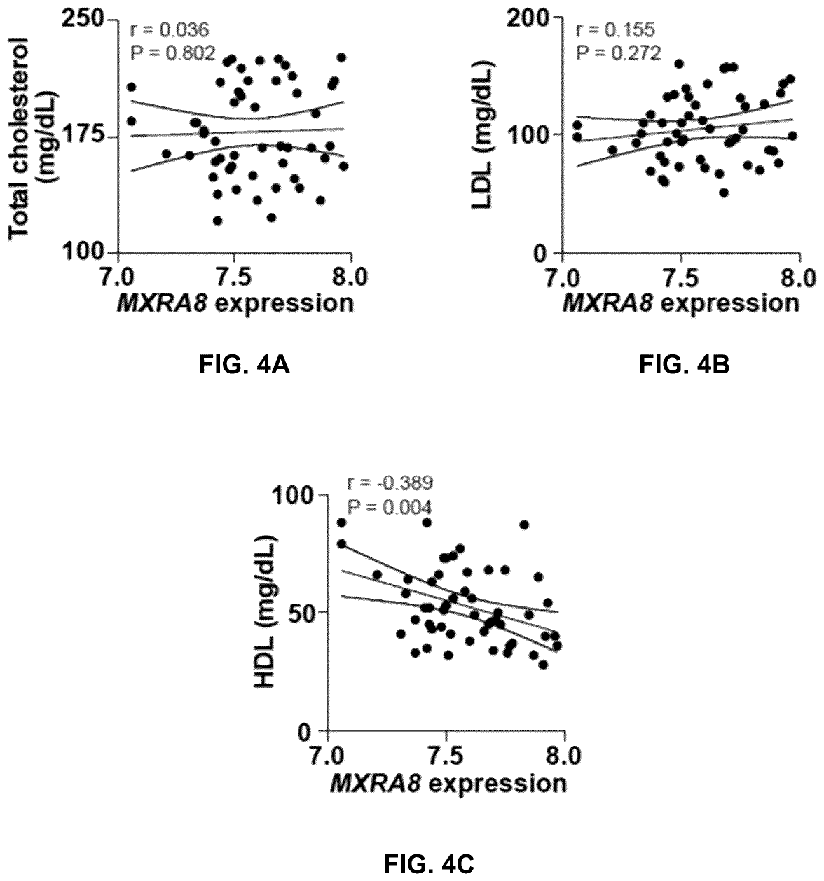

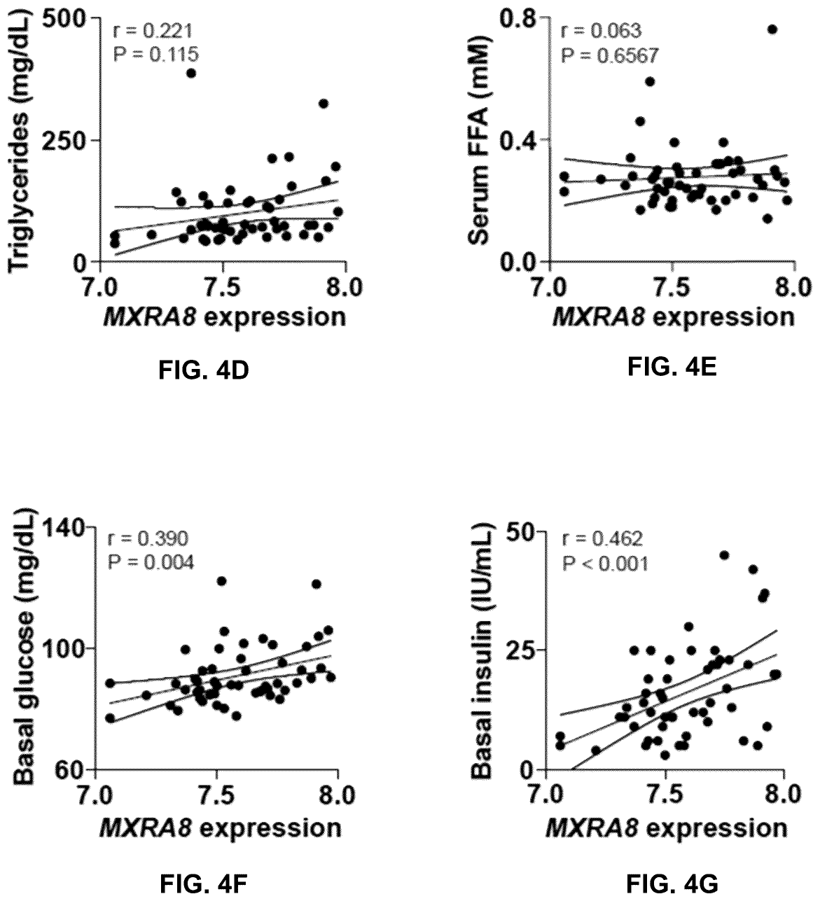

MXRA8 was more highly expressed in WAT of MAO patients than MNO counterparts (FIG. 2A), suggesting that MXRA8 expression may be related to the severity of metabolic disease. Consistent with this, MXRA8 expression was positively correlated with body mass index (BMI, FIG. 2C) as well as whole-body adiposity (FIG. 2D), subcutaneous adipose tissue volume (FIG. 2E), intra-abdominal adipose tissue volume (FIG. 2F), and liver fat content (FIG. 2G), anthropomorphic characteristics that were determined using dual-energy X-ray absorptiometry. MXRA8 expression was not correlated with lean mass (FIG. 2H) or bone mass (FIG. 2I). Although MXRA8 expression did not appear to correlate with total cholesterol or low-density lipoprotein (LDL) concentrations in plasma (FIG. 4A, FIG. 4B), higher expression of MXRA8 in WAT was correlated with lower concentrations of plasma high-density lipoprotein (HDL, FIG. 4C). Total serum triglycerides (FIG. 4D) and free fatty acid (FFA) concentrations also did not correlate with MXRA8 expression in WAT.

However, blood glucose and insulin concentrations in the morning prior to eating were positively correlated with MXRA8 expression in WAT (FIG. 4F, FIG. 4G). Furthermore, hyperglycemic patients exhibited higher WAT MXRA8 expression than those with euglycemia (FIG. 2J). These observations suggested a potential relationship between MXRA8 and glucose homeostasis. Indeed, higher MXRA8 expression in WAT was associated with impaired oral glucose tolerance (FIG. 2K) and more severe insulin resistance as indicated by hyperinsulinemic-euglycemic clamp studies (FIG. 2L). Taken together, these data indicate that MXRA8 expression is upregulated in WAT in obesity and that higher WAT MXRA8 expression is associated with impaired glucose metabolism in humans.

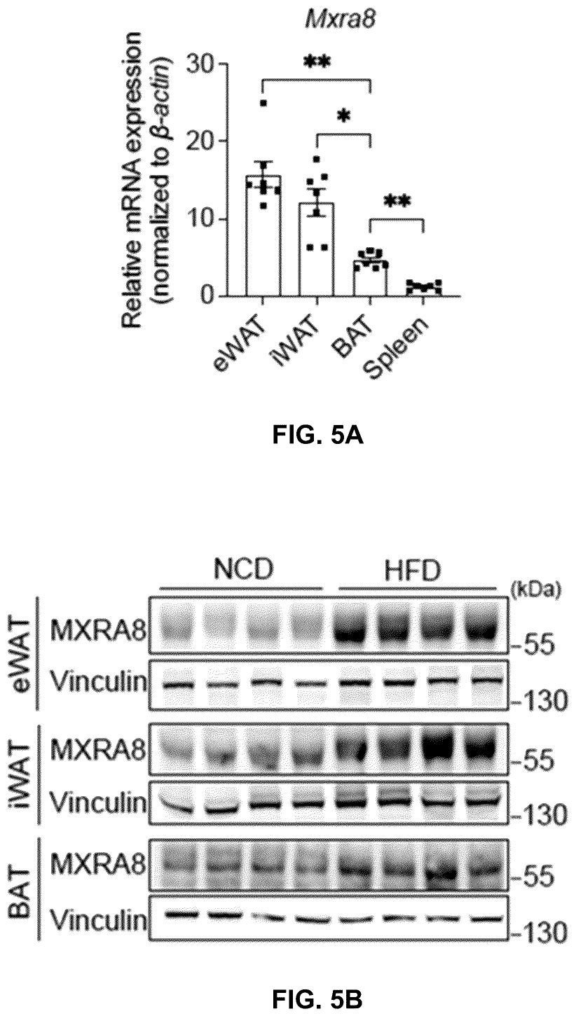

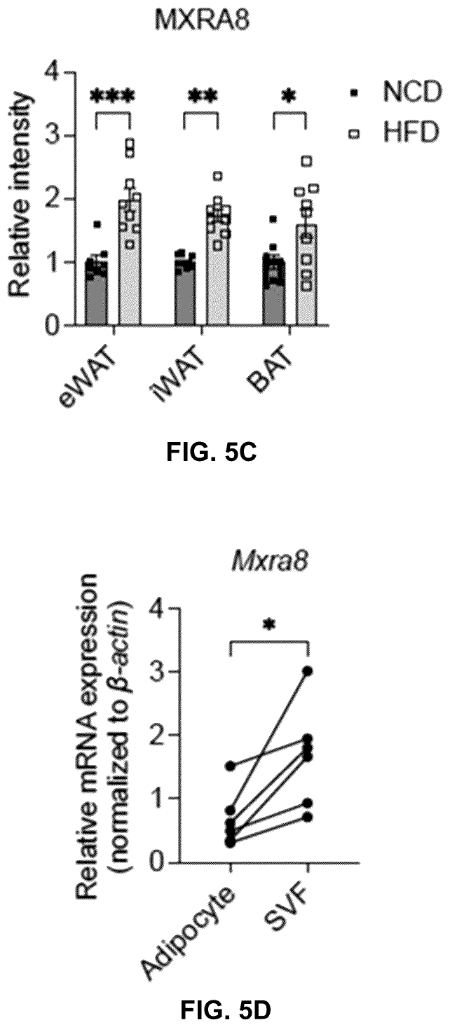

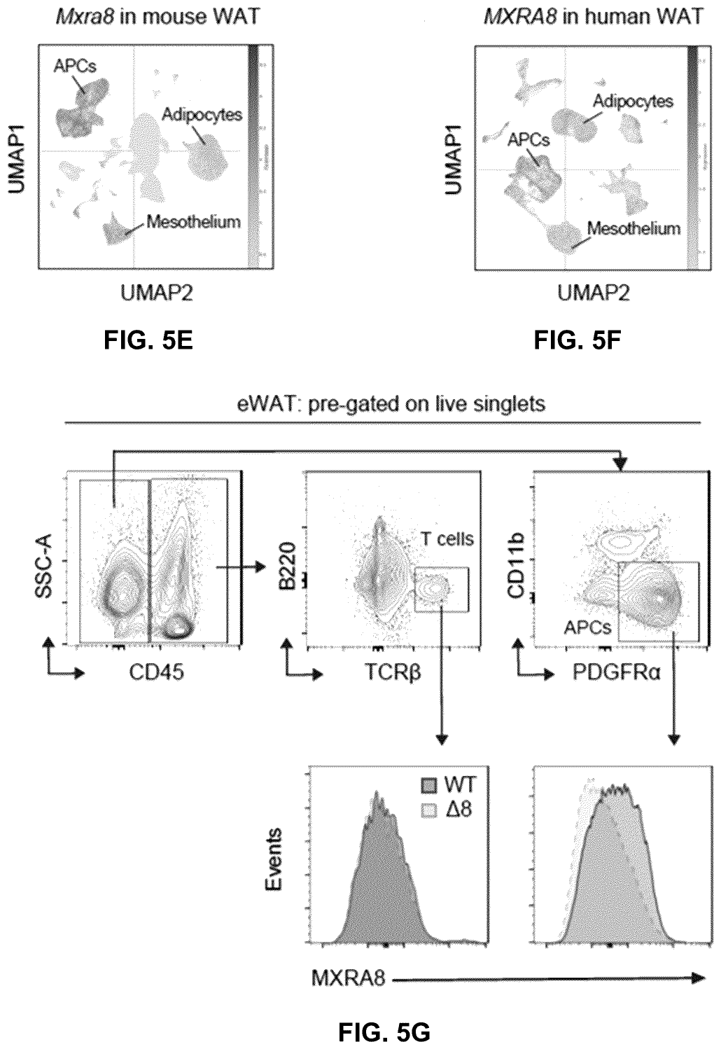

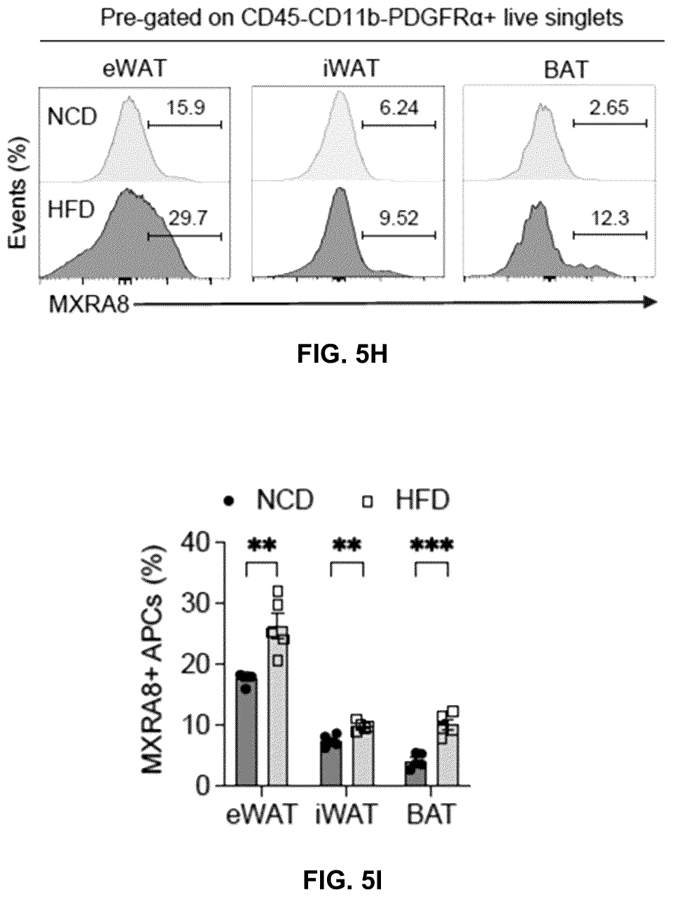

To study the functional role of MXRA8 in adipose tissue, the expression of MXRA8 was characterized in mice. Murine epididymal (e)WAT and inguinal (i)WAT expressed significantly higher Mxra8 transcript levels than did brown adipose tissue (BAT), and all three adipose depots had substantially higher levels of Mxra8 than did the spleen (FIG. 5A). As observed in human WAT, it was found that wildtype (WT) mice fed a high fat diet (HFD) for 10 weeks had upregulated MXRA8 protein expression in eWAT, iWAT, and BAT (FIG. 5B, FIG. 5C). This result indicates that upregulation of MXRA8 expression is a conserved characteristic of obesity in mice and humans.

To determine the cell types that express MXRA8, eWAT adipocytes and stromal vascular fraction (SVF) cells were first isolated from WT mice fed a normal chow diet and observed significantly higher expression of Mxra8 in the SVF than adipocytes (FIG. 5D). The SVF contains numerous cell types including all immune cell lineages and adipocyte progenitor cells, therefore publicly available single nucleus RNA sequencing (snRNAseq) datasets (GSE176171) were used and it was found that expression of MXRA8 mRNA was highest in Adipocyte Progenitor Cells (APCs) in both mice (FIG. 5E) and humans (FIG. 5F). MXRA8 mRNA was also expressed by mature adipocytes and mesothelial cells to lesser degrees in both species (FIG. 5E, FIG. 5F). To verify expression of MXRA8 protein on APCs, flow cytometry of eWAT SVF from WT vs MXRA8-deficient mice (Mxra8d8/d8) was performed and it was found that APCs but not T cells were MXRA8+(FIG. 5G). Furthermore, expression of MXRA8 was upregulated on APCs in eWAT, iWAT, and BAT harvested from HFD-induced obese mice compared to chow controls (FIG. 5H, FIG. 5I).

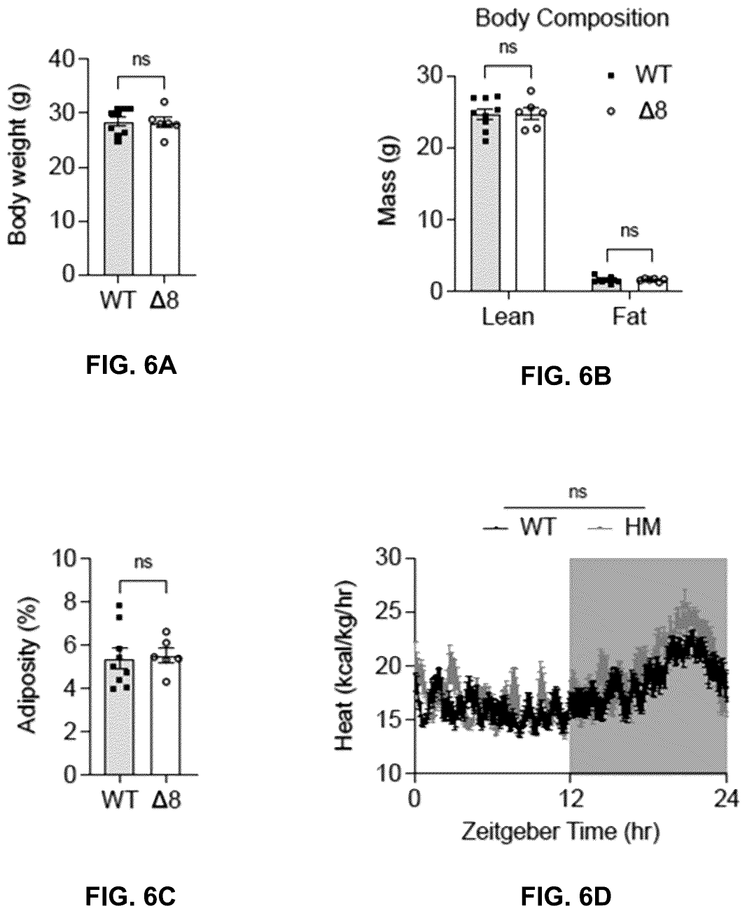

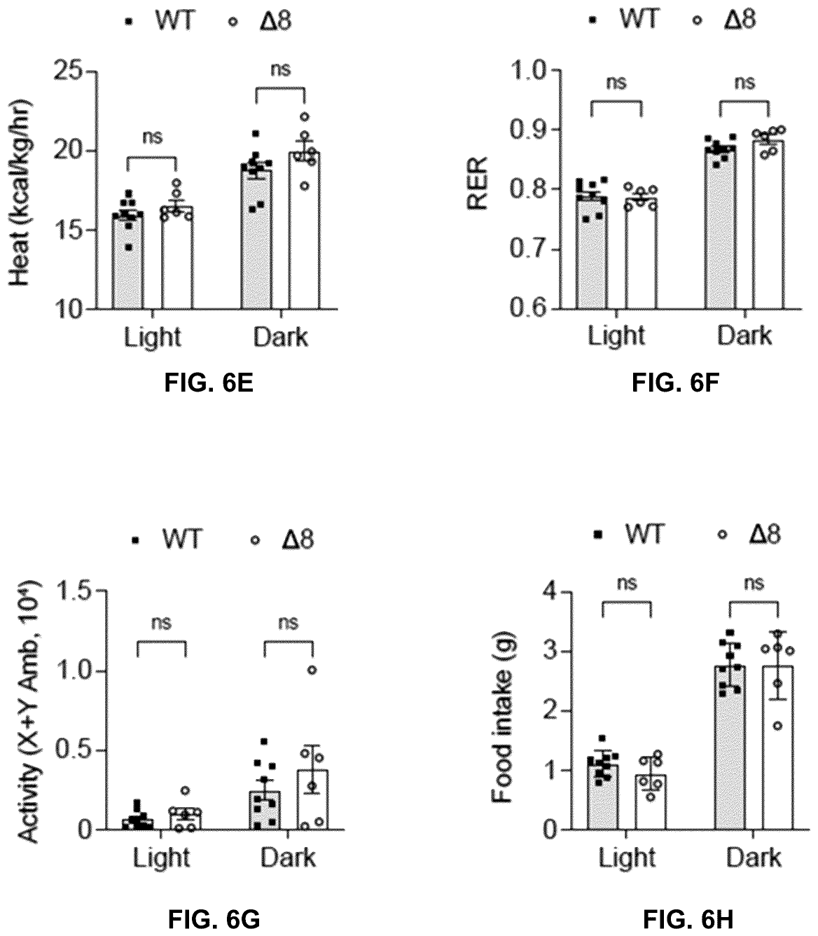

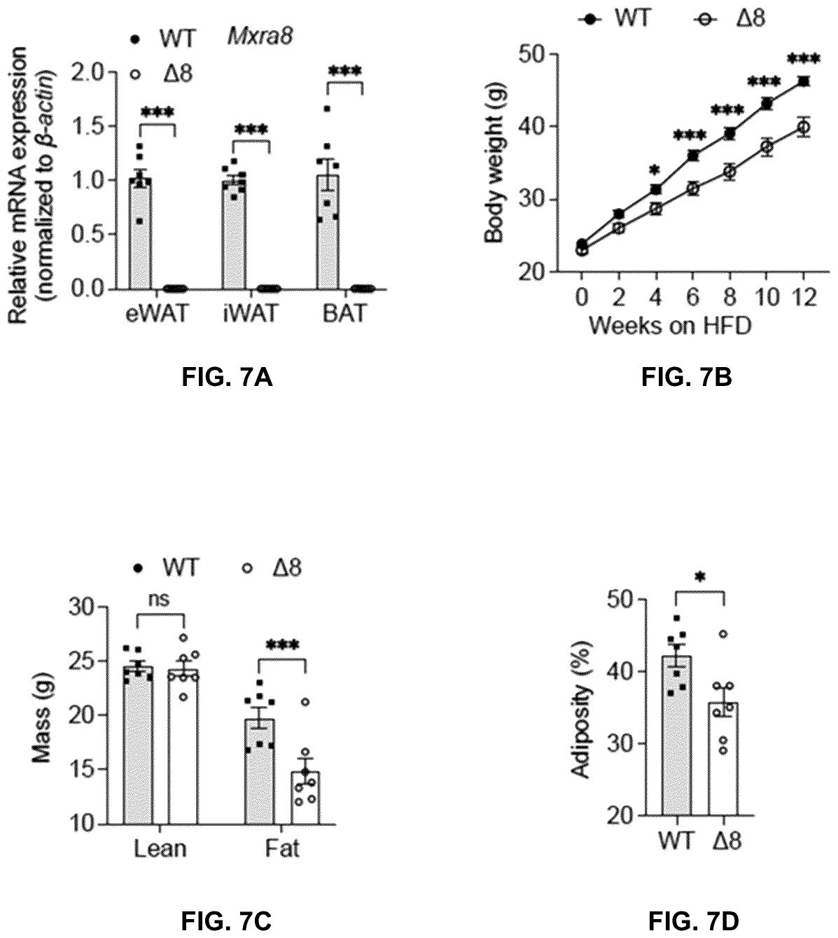

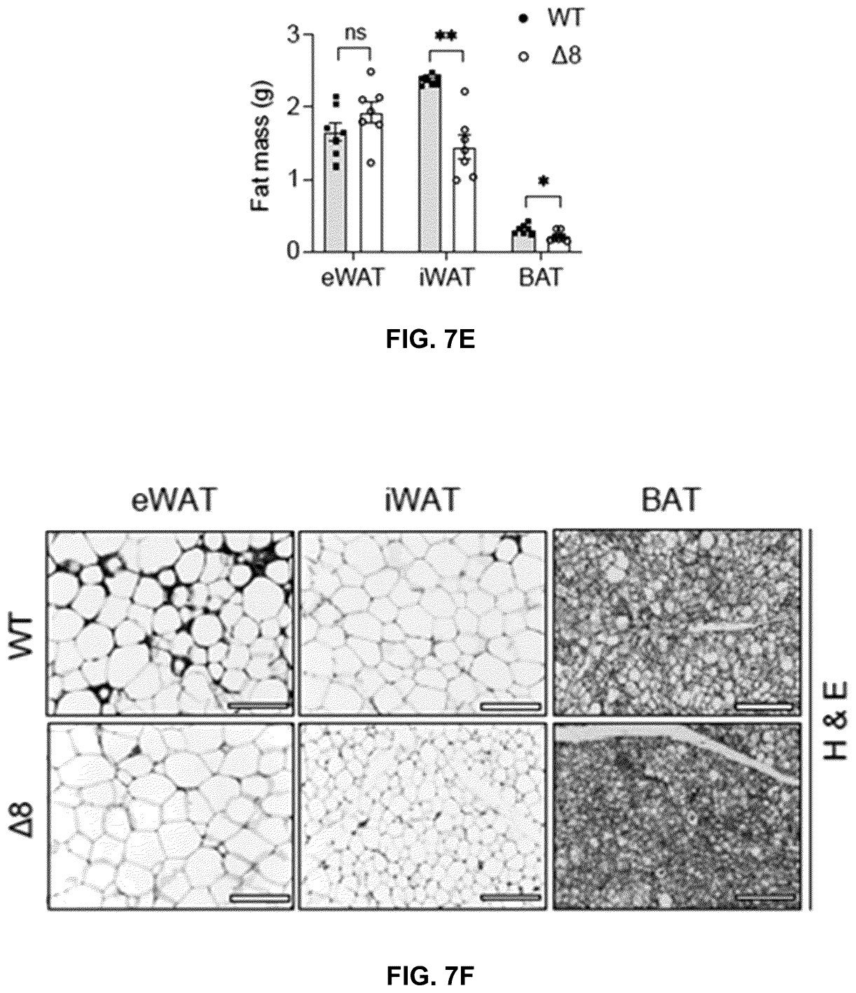

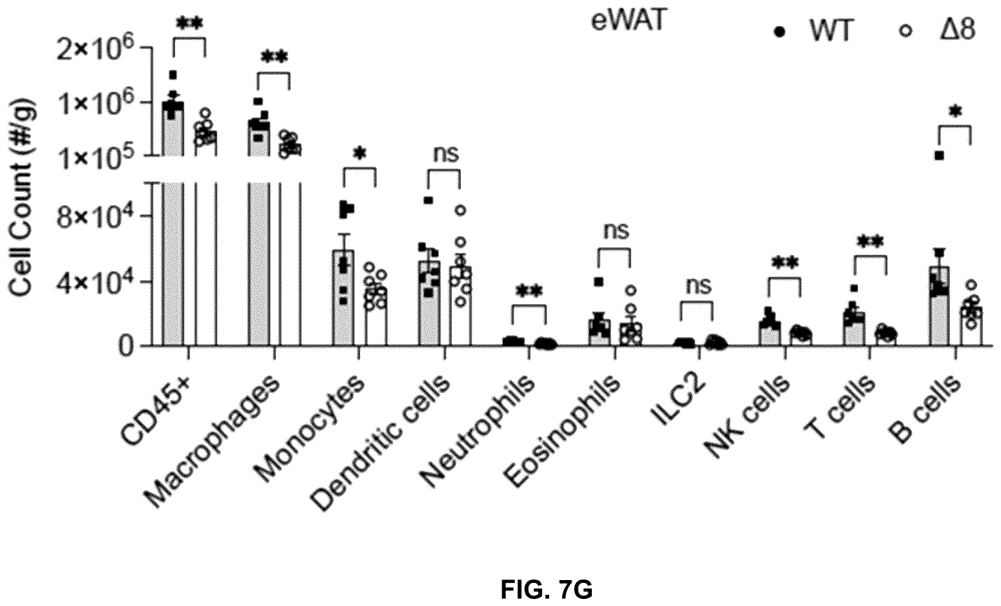

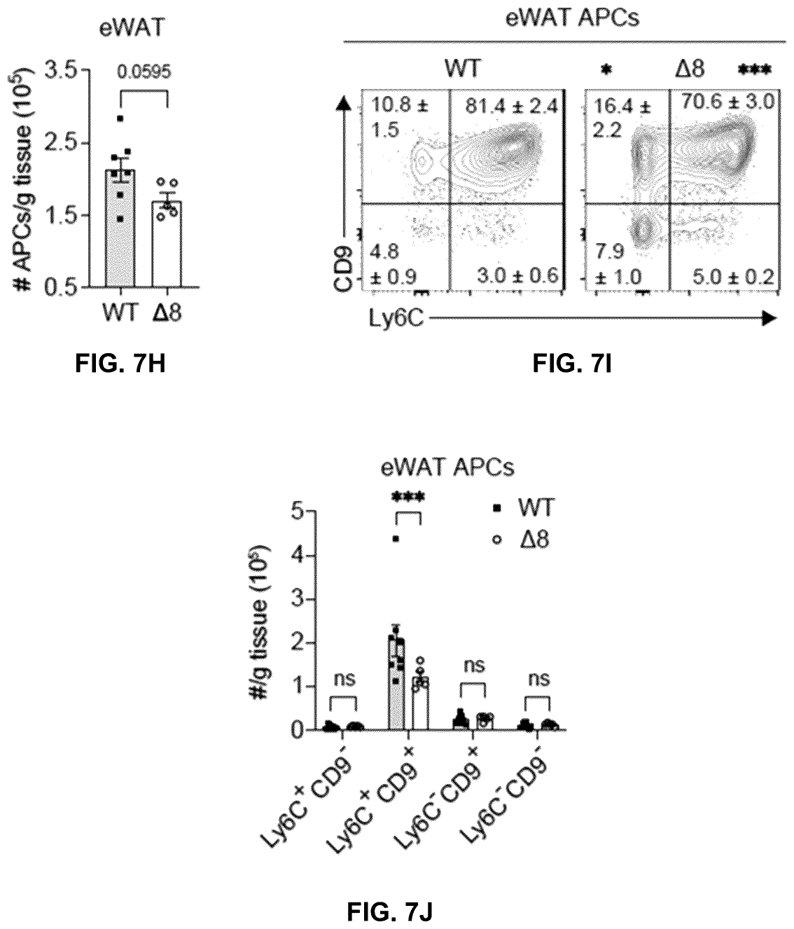

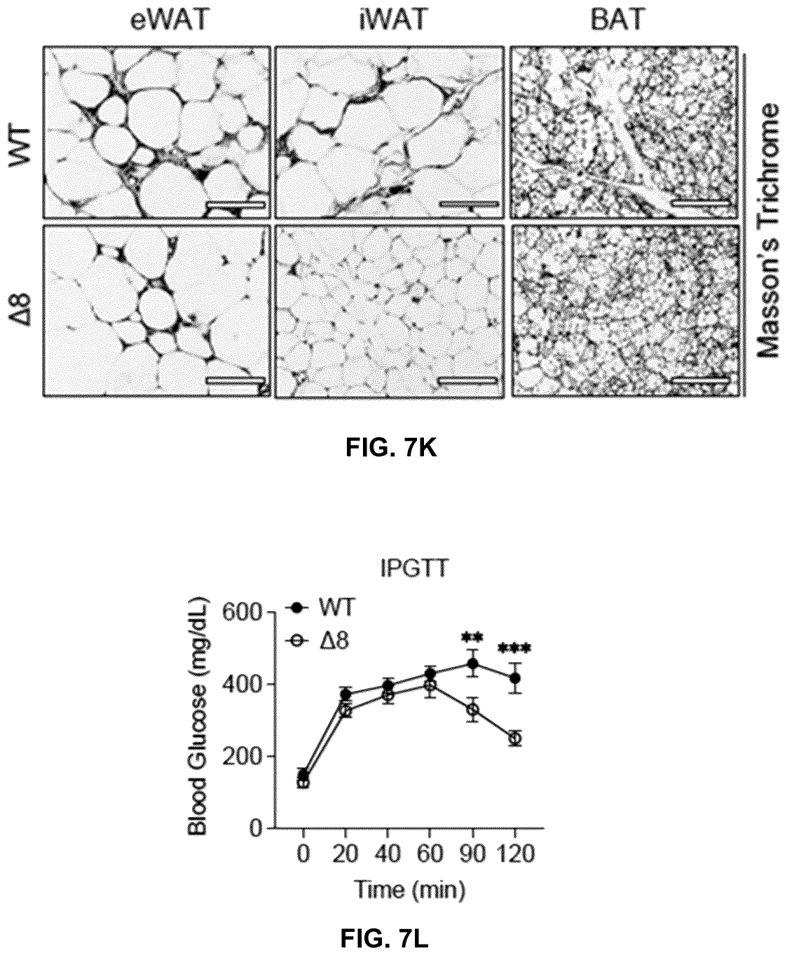

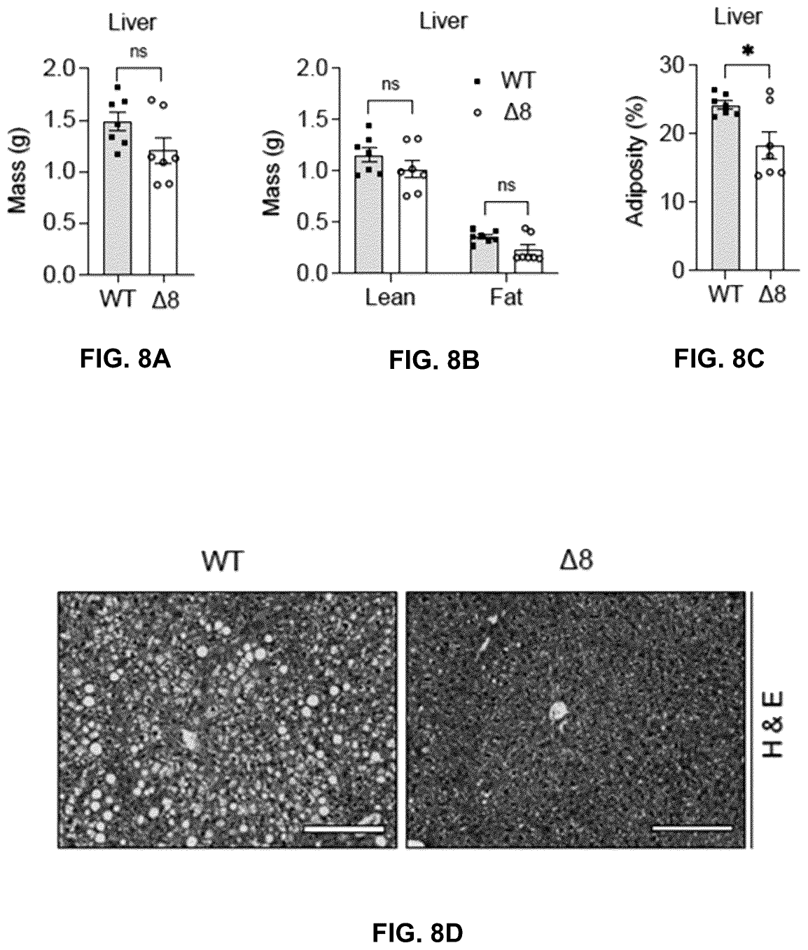

Next, the metabolic characteristics of MXRA8-deficient mice were compared to WT littermate controls. Deletion of Mxra8 mRNA in eWAT, iWAT, and BAT from Mxra8d8/d8 mice was first confirmed (FIG. 7A). Although these mice lack MXRA8 on their surface, they do produce a secreted form of MXRA8 with no known functional effects. On a normal chow diet WT and Mxra8d8/d8 mice had similar body weights, lean mass, fat mass, and adiposity (FIG. 6A, FIG. 6B, FIG. 6C), and metabolic cage analyses indicated the two genotypes had similar energy expenditure, respiratory exchange ratios, activity levels, and food intake (FIG. 6E, FIG. 6F, FIG. 6G, FIG. 6H). However, it was found that Mxra8d8/d8 mice gained less weight (FIG. 7B) and accumulated less whole-body fat mass and adiposity (FIG. 7C, FIG. 7D) than WT controls when challenged with a HFD. eWAT mass did not differ between groups, yet iWAT and BAT masses were significantly lower in Mxra8d8/d8 mice (FIG. 7E), suggesting that MXRA8 may have tissue-specific effects on thermogenic adipose tissues. To assess whether the lipid was being stored in ectopic sites rather than adipocytes, the liver was examined and observed significantly lower liver fat accumulation (FIG. 8A, FIG. 8B, FIG. 8C, FIG. 8D), suggesting that the reduction in iWAT and BAT masses in Mxra8d8/d8 mice was not associated with abnormal redistribution of lipids in ectopic sites such as the liver.

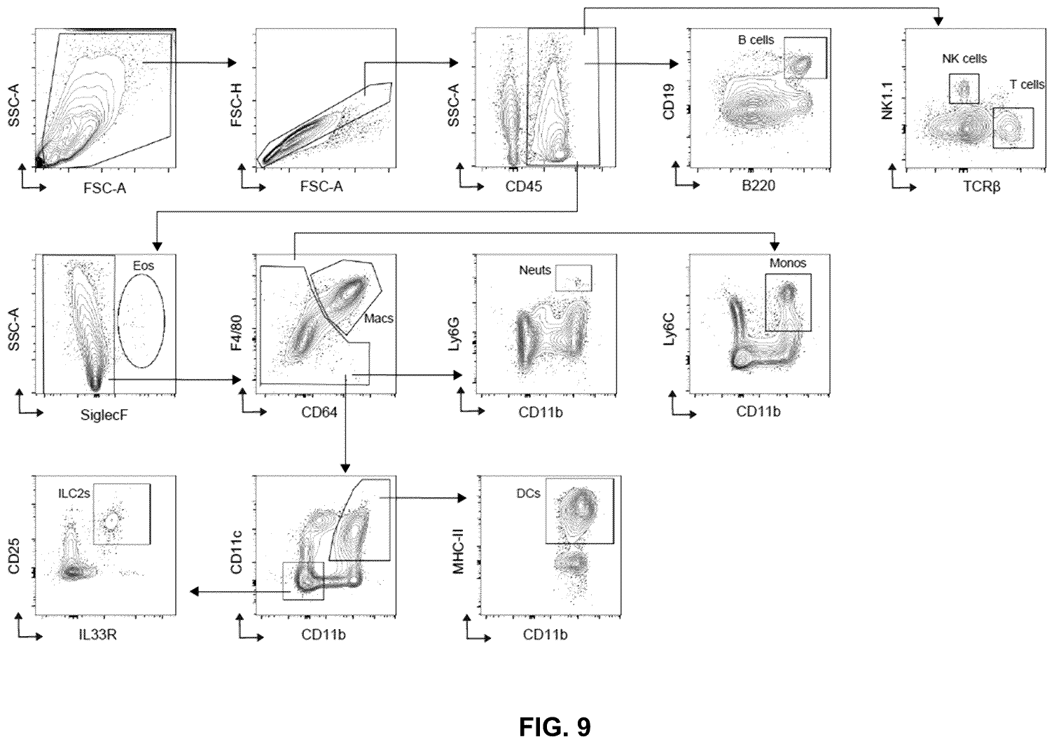

Histologic analyses of eWAT, iWAT, and BAT revealed several prominent features. In eWAT, there were no apparent differences in adipocyte size between groups, which is consistent with eWAT masses being similar. However, there were numerous crown-like structures (CLS) in eWAT from WT mice, but not Mxra8d8/d8 mice fed a HFD (FIG. 7F). CLS are highly enriched in immune cells and are strongly correlated with WAT inflammation in obesity (REFS). High-dimensional spectral flow cytometric analyses confirmed that eWAT from Mxra8d8/d8 mice had substantially fewer total immune cells, macrophages, monocytes, neutrophils, natural killer/group 1 innate lymphoid cells, T cells and B cells compared to WT controls, whereas eosinophil and group 2 innate lymphoid cell (ILC2) abundances did not differ (FIG. 7G and FIG. 9). In iWAT, there were relatively few CLS in both genotypes, and the adipocytes were smaller in Mxra8d8/d8 compared to WT mice (FIG. 7F), consistent with the morphologic changes seen in beiging or browning of WAT. A similar phenotype was observed in BAT of Mxra8d8/d8 versus WT mice (FIG. 7F).

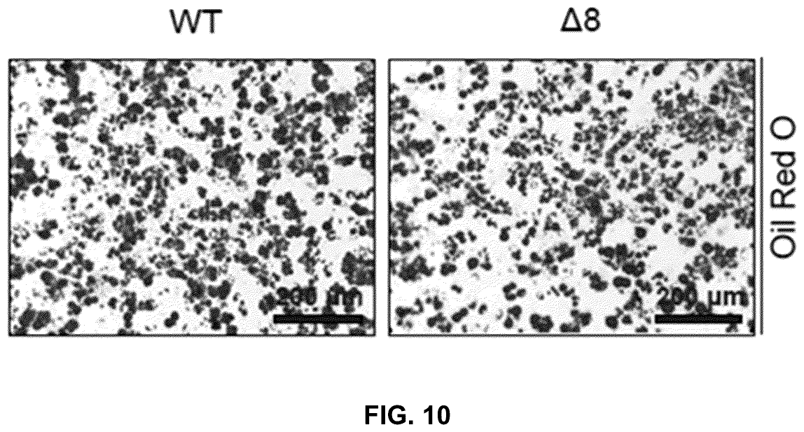

Recent studies suggest that a Ly6C+CD9+ APC subset known as fibroinflammatory progenitors (FIPs) produce numerous factors that drive inflammation in obese WAT and also contribute to fibrotic remodeling of WAT. This process is considered to be pathologic, and previous studies indicate that the degree of WAT fibrosis is directly correlated with WAT inflammation and metabolic abnormalities in obese mice and humans, including impaired glucose homeostasis. Furthermore, as noted above, APCs express MXRA8 and upregulate levels of this protein in obesity (FIG. 5E, FIG. 5F, FIG. 5G, FIG. 5H, FIG. 5I). the total numbers of APCs per gram of eWAT (FIG. 7H) and relative abundance of Ly6C+CD9+ FIPs in eWAT were observed to be lower in Mxra8d8/d8 versus WT mice following HFD feeding (FIG. 7I, FIG. 7J). These findings were associated with reduced fibrosis in eWAT, iWAT, and BAT, as indicated by Masson's trichrome staining (FIG. 7K) and improved glucose tolerance (FIG. 7L) in mice lacking MXRA8 compared to WT controls. However, the adipogenic potential of adipocytes did not appear to be markedly different in WT and Mxra8d8/d8 mice, as eWAT primary adipocytes accumulated Oil Red O-staining lipid droplets similarly in both genotypes (FIG. 10). These data suggest that MXRA8 might contribute to regulating APC responses in adipose tissue and promoting the inflammation and fibrosis that occur in obese WAT. However, it is also possible that these observed phenotypes are explained by the reduction in weight gain in the absence of MXRA8.

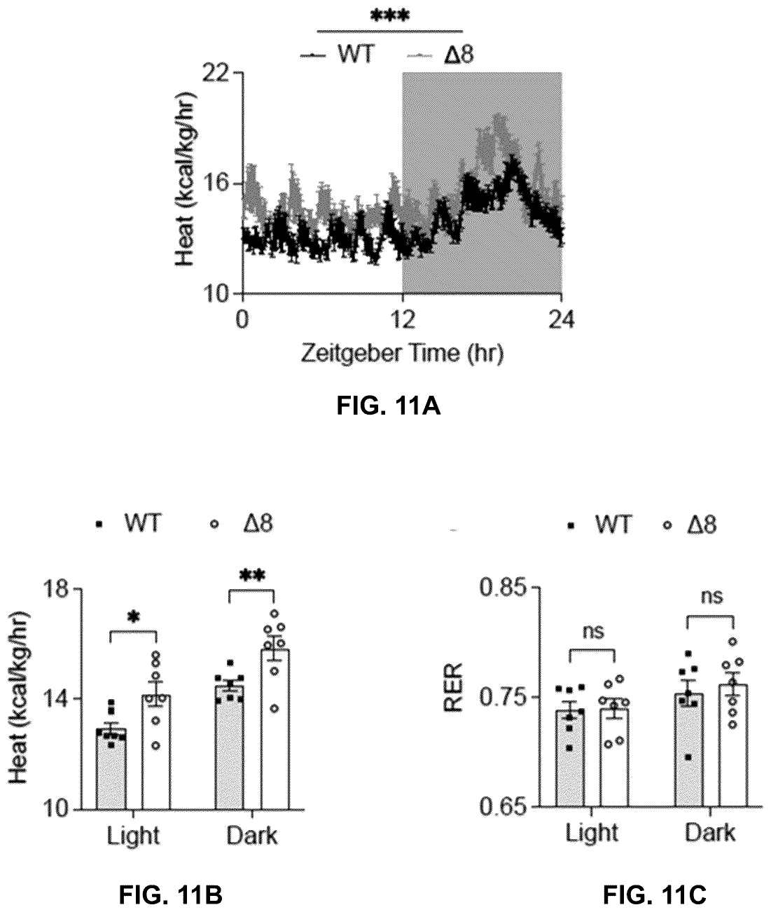

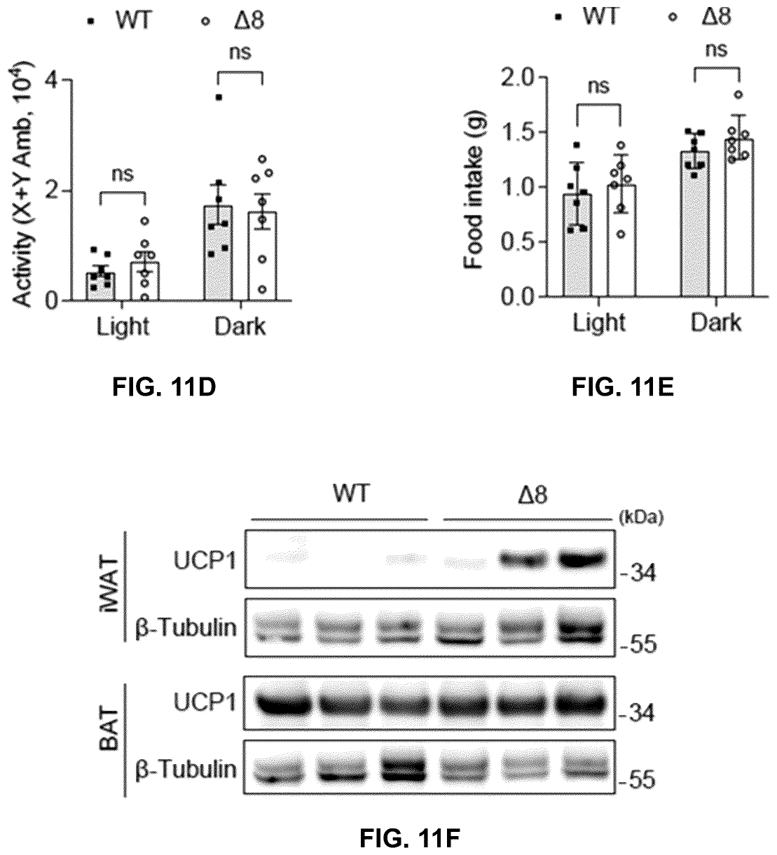

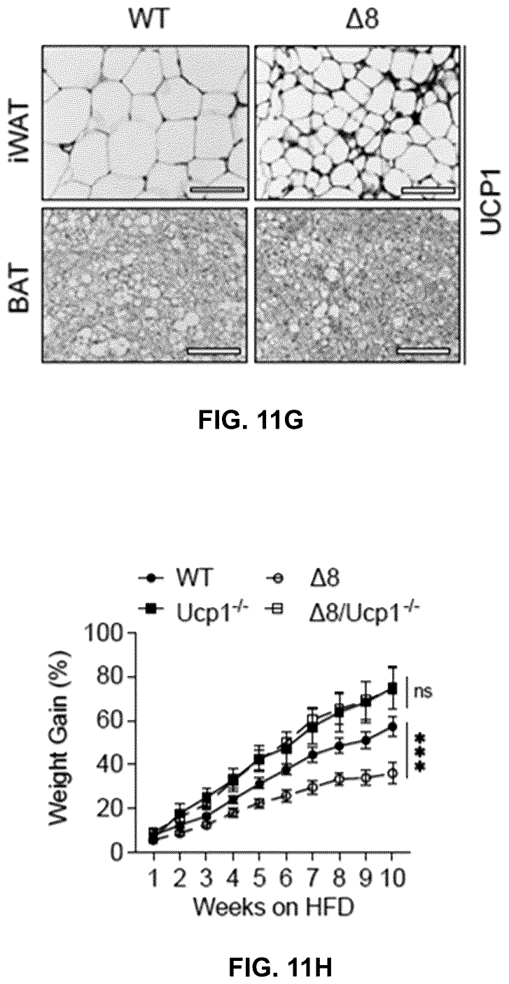

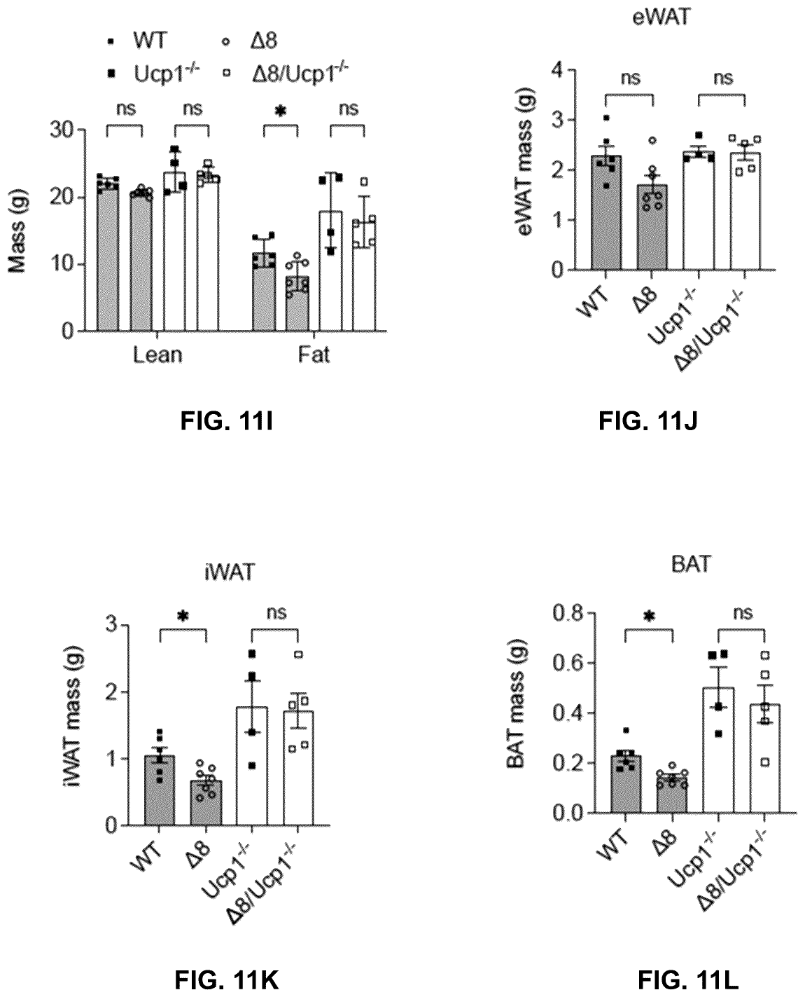

To investigate the mechanisms by which MXRA8 deficiency protects mice from HFD-induced obesity, WT and Mxra8d8/d8 mice fed a HFD were housed in metabolic cages. It was observed that Mxra8d8/d8 mice had significantly higher energy expenditure in both the light and dark phases of the light:dark cycle (FIG. 11A, FIG. 11B). There were no differences in RER, suggesting that there were no differences in energy substrate utilization (FIG. 11C). In addition, there were no significant alterations in activity levels (FIG. 11D) or food intake (FIG. 11E). These data suggest that MXRA8-deficient mice might have increased thermogenesis. Consistent with this possibility, Mxra8d8/d8 mice tended to exhibit increased expression of Uncoupling protein 1 (UCP1) in iWAT and BAT based on Western Blots (FIG. 11F) and immunohistochemistry (FIG. 11G).

Next, to test whether UCP1-dependent thermogenesis is required for the weight-gain protection in MXRA8-deficient mice, double knockouts that lack expression of both MXRA8 and UCP1 were generated. WT, Ucp1−/−, Mxra8d8/d8, and Ucp1−/−; Mxra8d8/d8 mice were housed at thermoneutrality for 2 weeks prior to initiating a HFD. It was found that Ucp1−/− mice gain more weight than WT controls in response to a HFD (FIG. 11H), consistent with prior reports. Strikingly, although Mxra8d8/d8 mice are still protected from weight gain at thermoneutrality, there were no differences in weight gain between Ucp1−/− and Ucp1−/−; Mxra8d8/d8 mice (FIG. 11H). Body composition analyses further indicated that Mxra8d8/d8 mice accumulated less whole-body fat than WT mice, but this effect was ablated in the absence of UCP1 (FIG. 11I). Similar trends were observed for eWAT, iWAT and BAT masses (FIG. 11J, FIG. 11K, FIG. 11L). Collectively, these data indicate that MXRA8 deficiency protects against HFD-induced weight gain in a UCP1-dependent manner.

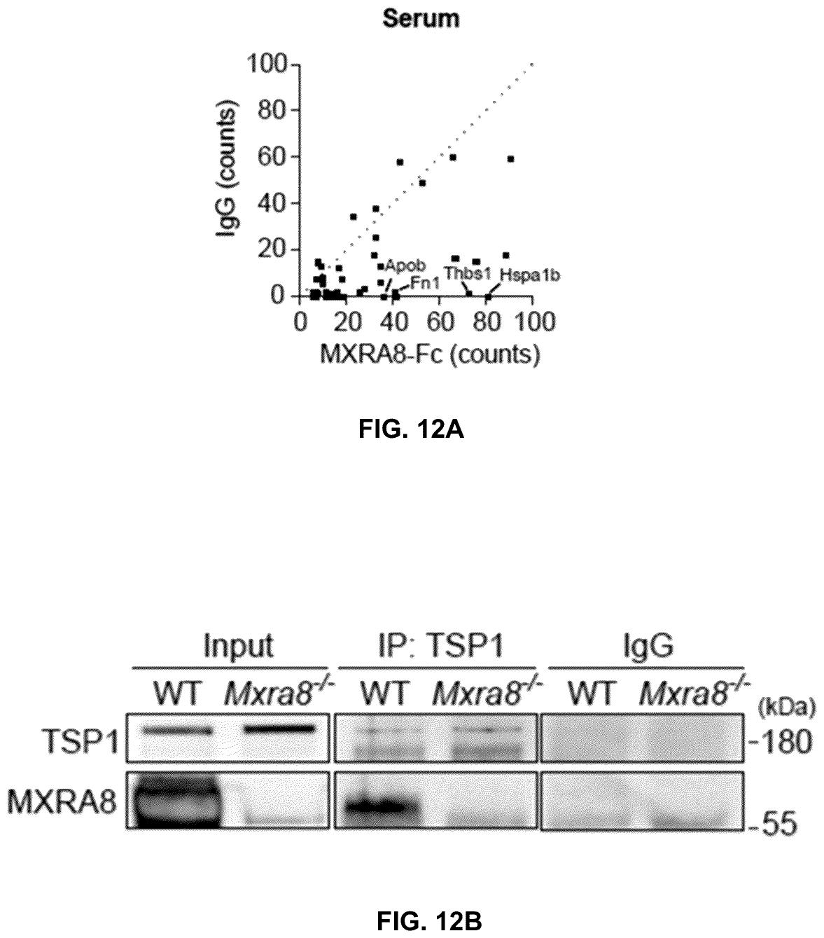

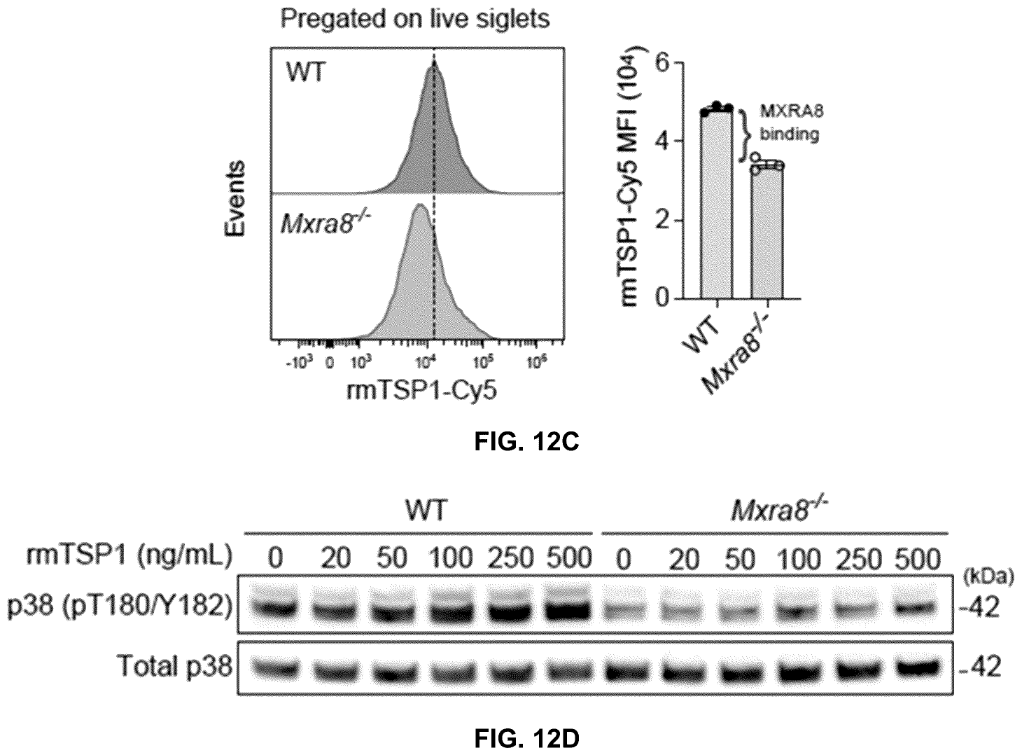

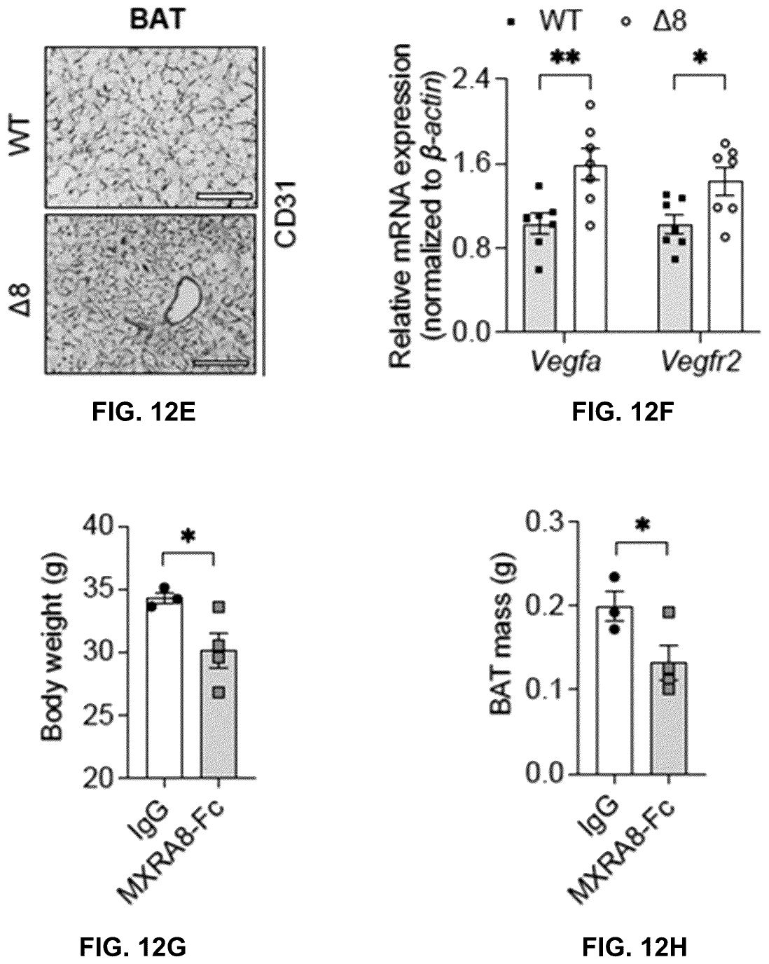

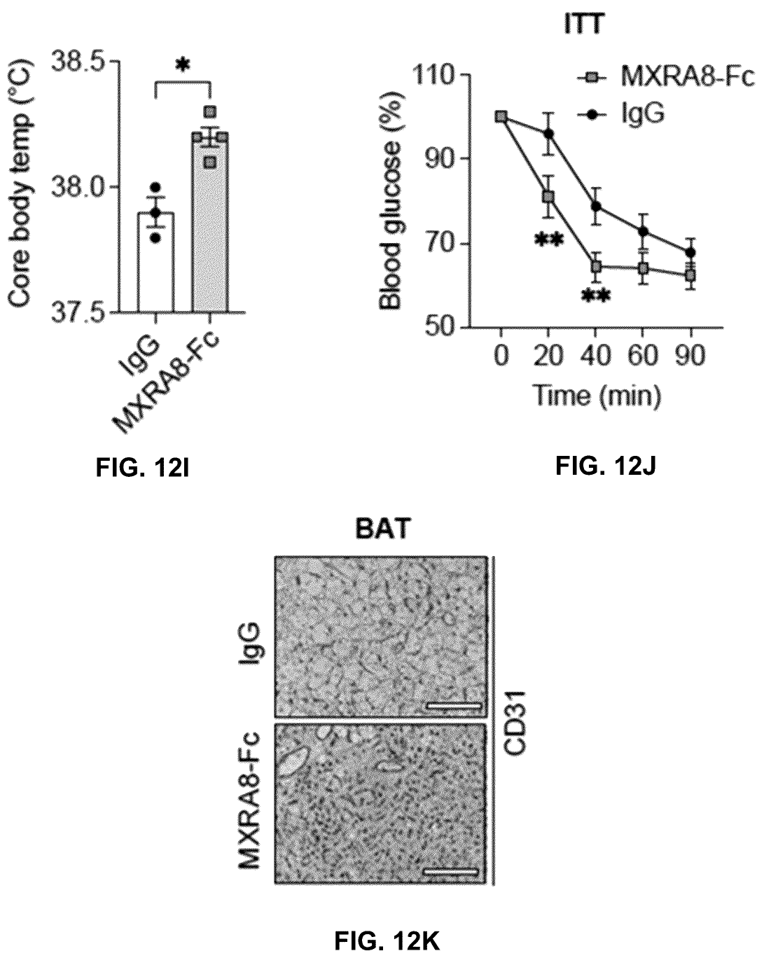

To determine the mechanisms by which MXRA8 limits obesity pathogenesis, a synthetic MXRA8-Fc fusion protein was generated and attached to protein A-coated Dynabeads. The MXRA8-coated beads were then incubated with serum from MXRA8-deficient mice fed a HFD to immunoprecipitate bound proteins for identification by mass spectrometry proteomics. This experiment revealed that MXRA8 binds an anti-angiogenic protein called Thrombospondin-1 (TSP1, also known as Thbs1; FIG. 12A). To verify this interaction in cells, TSP1 in WT or MXRA8-deficient 3T3 fibroblasts was immunoprecipitated and it was found that MXRA8 was pulled down by TSP1 (FIG. 12B). Also, recombinant mouse (rmTSP1) was labeled with the fluorescent dye Cy5 and found that TSP1 binds to WT cells with a higher mean fluorescence intensity than in MXRA8-deficient cells (FIG. 12C). Furthermore, low-dose recombinant mouse TSP1 induces signaling to p38 MAP kinase within 40 min in WT 3T3 fibroblasts but not in the absence of MXRA8 (FIG. 12D). Consistent with this interaction, Mxra8d8/d8 mice with a secreted form of MXRA8 were found to have increased vascular density (FIG. 12E) and increased expression of the pro-angiogenic genes Vegfa and Vegfr2 (FIG. 12F) in BAT compared to WT controls. Collectively, these data indicate that TSP1 is a natural ligand for MXRA8 and binds this receptor to inhibit angiogenesis.

Based on these findings, MXRA8-Fc fusion protein or IgG control antibody was administered to WT mice fed a HFD to neutralize TSP1/MXRA8 interactions in vivo. It was found that MXRA8-Fc treatment decreased weight gain compared to controls (FIG. 12G). This anti-obesity effect was associated with decreased BAT mass (FIG. 12 H) and increased core body temperature (FIG. 12I), which is supportive of increased adaptive thermogenesis. In addition, insulin resistance (FIG. 12.J) and vascular density (FIG. 12k) were improved with MXRA8-Fc therapy. These results indicate that biologic-based inhibition of MXRA8/TSP1 interactions reduces obesity and is a novel therapeutic strategy to treat obesity and associated metabolic diseases such as type 2 diabetes.

Overall, these data support a model in which TSP1 binds to MXRA8 to inhibit the vascular bed in obesity, leading to failure of thermogenic beige/brown adipocytes and decreased energy expenditure. This favors weight gain to promote obesity pathogenesis. A synthetic MXRA8-Fc fusion protein that binds soluble TSP1 preserves the vascular bed in adipose tissue and prevents HFD-induced obesity. These findings reveal that therapeutically targeting MXRA8 is a novel strategy to treat obesity and associated metabolic diseases, such as type 2 diabetes.

What is claimed is:

1. A composition comprising a synthetic MXRA8-Fc fusion protein or pharmaceutically acceptable salt thereof.

2. The composition of claim 1, wherein the synthetic MXRA8-Fc fusion protein comprises an immunoglobulin (IgG) Fc domain and an MXRA8 protein.

3. The composition of claim 1, further comprising at least one of a pharmaceutically acceptable excipient and a pharmaceutically acceptable carrier.

4. The composition of claim 1, further comprising protein A-coated beads attached to the synthetic MXRA8-Fc fusion protein.

5. A method of treating a metabolic associated disease in a subject in need thereof, the method comprising:
administering to the subject a composition comprising a synthetic MXRA8-Fc fusion protein or pharmaceutically acceptable salt thereof.

6. The method of claim 5, wherein administering the composition neutralizes TSP1/MXRA8 interactions in the subject.

7. The method of claim 5, wherein the synthetic MXRA8-Fc fusion protein comprises an immunoglobulin (IgG) Fc domain and an MXRA8 protein.

8. The method of claim 5, further comprising at least one of a pharmaceutically acceptable excipient and a pharmaceutically acceptable carrier.

9. The method of claim 5, further comprising protein A-coated beads attached to the synthetic MXRA8-Fc fusion protein.

10. The method of claim 5, wherein the metabolic associated disease is a high fat diet (HFD)-induced disease.

11. The method of claim 10, wherein the HFD-induced disease is type 2 diabetes.

12. The method of claim 10, wherein the HFD-induced disease is fatty liver disease.

13. A method of treating obesity in a subject in need thereof, the method comprising:
administering to the subject a composition comprising a synthetic MXRA8-Fc fusion protein or pharmaceutically acceptable salt thereof.

14. The method of claim 13, wherein administering the composition neutralizes TSP1/MXRA8 interactions in the subject.

15. The method of claim 13, wherein the synthetic MXRA8-Fc fusion protein comprises an immunoglobulin (IgG) Fc domain and an MXRA8 protein.

16. The method of claim 13, further comprising at least one of a pharmaceutically acceptable excipient and a pharmaceutically acceptable carrier.

17. The method of claim 13, further comprising protein A-coated beads attached to the synthetic MXRA8-Fc fusion protein.

18. The method of claim 13, wherein the obesity is high fat diet (HFD)-induced obesity.

19. The method of claim 18, wherein the subject has type 2 diabetes.

20. The method of claim 18, wherein the subject has fatty liver disease.
